(19) United States
(12) Patent Application Publication
(10) Pub. No.: US 2024/0041926 A1
(43) Pub. Date: Feb. 8, 2024

(54) DENDRITIC CELL ACTIVATING CHIMERIC ANTIGEN RECEPTORS AND USES THEREOF

(71) Applicants: SHENZHEN FRONTIERGATE BIOTECHNOLOGY CO., LTD, SHENZHEN, GUANGDONG (CN); GUANGDONG SANSCI BIOTECHNOLOGY CO., LTD, GUANGDONG (CN)

(72) Inventor: Yang XU, SHENZHEN, GUANGDONG (CN)

(73) Assignees: SHENZHEN FRONTIERGATE BIOTECHNOLOGY CO., LTD, SHENZHEN, GUANGDONG (CN); GUANGDONG SANSCI BIOTECHNOLOGY CO., LTD, GUANGDONG (CN)

(21) Appl. No.: 18/265,498

(22) PCT Filed: Dec. 24, 2021
(86) PCT No.: PCT/CN2021/141311

§ 371 Date: Jun. 6, 2023

(30) Foreign Application Priority Data
Jan. 8, 2021 (CN) 202110022268.5

(51) Int. Cl. A61K 35/17; C07K 14/705; C12N 5/0783; C07K 16/28; C12N 5/0784; A61P 35/00; A61K 39/00; C12N 15/86

(52) U.S. Cl. CPC A61K 35/17; C07K 14/70517; C12N 5/0636; C07K 16/2803; C07K 16/2866; C12N 5/0639; A61P 35/00; A61K 39/4611; A61K 39/4631; A61K 39/464422; A61K 39/464429; C12N 15/86; A61K 2239/13; A61K 2239/17; A61K 2239/21; A61K 2239/22

(57) ABSTRACT

The present disclosure provides a chimeric antigen receptor (CAR) for activating dendritic cells (DCs) in an immunosuppressive tumor environment. The present disclosure also provides compositions comprising the CAR, polynucleotides encoding the CAR, vectors comprising a polynucleotide encoding the CAR, engineered cells comprising the CAR, and method using the same.

### CROSS-REFERENCE TO RELATED APPLICATIONS

This application claims the priority to Chinese patent application no. 202110022268.5 filed Jan. 8, 2021, the entire disclosure of which is incorporated herein by reference.

### FIELD OF THE INVENTION

The present disclosure generally relates to the field of cell therapy. In particular, the present disclosure relates to compositions and methods for activating dendritic cells (DCs) in an immune suppressive tumor microenvironment.

### BACKGROUND

As a key link between innate and adaptive immune systems, dendritic cells (DCs) are the major antigen presenting cells (APCs) to activate T cell-dependent immunity (R. M Steinman, Decisions about dendritic cells: past, present, and future. Annu. Rev. Immunol. 30, 1-22 (2012); and S. Puhr et al., Dendritic cell development-History, advances, and open questions. Semin. Immunol. 27, 388-396 (2015)), especially in triggering tumor-specific immune responses (M Hansen et al., The role of dendritic cells in cancer. Semin. Immunopathol. 39, 307-316 (2017)). Previous studies have revealed that tumor-infiltrating dendritic cells (TIDCs) usually exhibit an immature or dysfunctional phenotype in immune suppressive tumor microenvironment or tumor immune suppressive microenvironment (TIME), which suppresses the infiltration and activation of T cells (J. M Tran Janco et al., Tumor-infiltrating dendritic cells in cancer pathogenesis. J. Immunol. 194, 2985-2991 (2015)).

While many signaling pathways have been identified for rescuing the aberrant behaviors of TIDCs, such as siRNA silencing of PD-L1 and PD-L2 on dendritic cells, no significant progress has been achieved in their clinical applications (W. Hobo et al., siRNA silencing of PD-L1 and PD-L2 on dendritic cells augments expansion and function of minor histocompatibility antigen-specific CD8+ T cells. Blood 116, 4501-4511 (2010); A. Harari et al., Antitumour dendritic cell vaccination in a priming and boosting approach. Nat. Rev. Drug Discovery 19, 635-652 (2020); and Y. Ma et al., Dendritic Cells in the Cancer Microenvironment. J. Cancer 4, 36-44 (2013)).

Therefore, need exists for developing a novel method for activating dendritic cells (e.g., tumor-infiltrating dendritic cells) in TIME.

### SUMMARY OF INVENTION

In one aspect, the present disclosure provides a polynucleotide encoding a chimeric antigen receptor (CAR), wherein the CAR comprising (1) an extracellular antigen-binding domain, (2) a transmembrane domain and (3) an intracellular signaling domain, wherein the CAR is capable of activating dendritic cells in an immune suppressive tumor microenvironment.

In certain embodiments, the immune suppressive tumor microenvironment comprises a tumor and/or tumor infiltrating immune cells that are: 1) expressing an immune inhibitory molecule, and/or 2) deficient in an immune stimulating cytokine.

In certain embodiments, the immune inhibitory molecule is selected from the group consisting of PD-1, TIM-3, TIGIT, LAG-3, A2AR, BTLA (CD272), CTLA-4 (CD152), IDO1, IDO2, TDO, NOX2, VISTA, SIGLEC7 (CD328), PVR(CD155) and SIGLEC9 (CD329), PD-L1, PD-L2, B7-H3 (CD276), B7-H4 (VTCN1), PVR(CD155), HLA class I, sialoglycoprotein, CD112, CD113, Galectin9, CD24, and CD47.

In certain embodiments, the immune inhibitory molecule is CTLA-4 and/or PD-L1.

In certain embodiments, the immune stimulating cytokine is selected from TNF-α, IFN-β, IFN-γ, IL-1, IL-2, IL-4, IL-6, IL-8, IL-10, IL-12, IL-18, granulocyte-macrophage colony stimulating factor and a combination thereof.

In certain embodiments, the tumor comprises a cell expressing CTLA4-Ig and/or PD-L1.

In certain embodiments, the immune suppressive tumor microenvironment comprises a tumor that has poor responsiveness to monotherapy of adoptive cell therapy (e.g., CAR-T monotherapy).

In certain embodiments, the intracellular signaling domain comprises a cytoplasmic domain of a dendritic cell activating receptor selected from the group consisting of RIG-1, NLRP10, DEC-205, BDCA-2, CD86, 4-1BBL, OX40L, CD40, IFNAR, TLR4, TNFR (e.g., TNFR2), CD80, CD40L, CD367 (DCIR), CD207 (Langerin), CD371 (DCAL-2, CLEC12a), CD204, CD36, IFNγR, Dectin-1 and FcγR, or a combination thereof.

In certain embodiments, the intracellular signaling domain comprises the cytoplasmic domain of Dectin-1 and the cytoplasmic domain of FcγR.

In certain embodiments, the cytoplasmic domain of Dectin-1 and the cytoplasmic domain of FcγR are connected in tandem.

In certain embodiments, the cytoplasmic domain of Dectin-1 comprises an amino acid sequence set forth in SEQ ID NO: 1, or any functional forms thereof.

In certain embodiments, the cytoplasmic domain of FcγR comprises an amino acid sequence set forth in SEQ ID NO: 2 or any functional forms thereof.

In certain embodiments, the intracellular signaling domain comprises an amino acid sequence set forth in SEQ ID NO: 3 or any functional forms thereof.

In certain embodiments, the intracellular signaling domain comprises an amino acid sequence encoded by a nucleic acid sequence set forth in SEQ ID NO: 4 or any functional forms thereof.

In certain embodiments, the extracellular antigen-binding domain comprises a single-chain variable fragment (scFv).

In certain embodiments, the scFv is specific for a tumor surface marker (e.g., solid tumor surface marker).

In certain embodiments, the tumor surface marker is selected from the group consisting of: EphA2, CD19, CD70, CD133, CD147, CD171, DLL3, EGFRvIII, Mesothelin, ganglioside GD2, FAP (fibroblast activating protein), FBP (folate binding protein), Lewis Y, Claudin 18.2, IL13Rα2, HER2, MDC1, PMSA (prostate membrane specific antigen), ROR1, B7-H3, CAIX, CD133, CD171, CEA, GPC3, MUC1, NKG2D.

In certain embodiments, the CAR further comprises a signal peptide.

In certain embodiments, the signal peptide comprises a signal peptide of CD8 alpha.

In certain embodiments, the signal peptide of CD8 alpha comprises a sequence set forth in SEQ ID NO: 5 or any functional forms thereof.

In certain embodiments, the transmembrane domain comprises a transmembrane domain of CD8 alpha.

In certain embodiments, the transmembrane domain of CD8 alpha comprises a sequence set forth in SEQ ID NO: 6, or any functional forms thereof.

In certain embodiments, the extracellular antigen-binding domain is linked to the transmembrane domain by a hinge region.

In certain embodiments, the hinge region comprises a hinge region of CD8 alpha.

In certain embodiments, the hinge region of CD8 alpha comprises a sequence set forth in SEQ ID NO: 7, or any functional forms thereof.

In certain embodiments, the polynucleotide provided herein is a DNA or RNA.

In another aspect, the present disclosure provides a polypeptide encoded by the polynucleotide provided herein.

In another aspect, the present disclosure provides a vector comprising the polynucleotide provided herein, wherein the polynucleotide encoding the CAR is operably linked to at least one regulatory polynucleotide element for expression of the CAR.

In certain embodiments, the vector is a plasmid vector, a viral vector, a transposon, a site directed insertion vector, or a suicide expression vector.

In certain embodiments, the viral vector is a lentiviral vector, a retroviral vector, or an AAV vector.

In certain embodiments, the viral vector is a lentiviral vector.

In another aspect, the present disclosure provides an engineered cell comprising the polypeptide provided herein.

In certain embodiments, the engineered cell is a dendritic cell or a precursor or progenitor cell thereof.

In certain embodiments, the dendritic cell or a precursor or progenitor cell thereof is derived from a peripheral blood cell, a bone marrow cell, an embryonic stem cell, or an induced pluripotent stem cell.

In another aspect, the present disclosure provides a method of producing the engineered cells provided herein, comprising introducing to a starting cell the vector provided herein under conditions suitable for expression of the polynucleotide provided herein.

In certain embodiments, the starting cell is a dendritic cell or a precursor or a progenitor cell thereof.

In certain embodiments, the dendritic cell or a precursor or a progenitor cell thereof is derived from a peripheral blood cell, a bone marrow cell, an embryonic stem cell, or an induced pluripotent stem cell.

In another aspect, the present disclosure provides a population of cells produced ex vivo by the method provided herein.

In certain embodiments, at least 70% of the population of cells express a detectable level of the polypeptide provided herein.

In another aspect, the present disclosure provides a pharmaceutical composition comprising (i) the polynucleotide provided herein, or the polypeptide provided herein, or the vector provided herein, or the population of the engineered cells provided herein or the population of cells provided herein, and (ii) a pharmaceutically acceptable medium.

In another aspect, the present disclosure provides a method for improving efficacy of adoptive cell therapy in treating cancer in a subject in need thereof, comprising administering a therapeutically effective amount of the pharmaceutical composition provided herein.

In certain embodiments, the adoptive cell therapy comprises adoptive transfer of modified immune cells.

In certain embodiments, the pharmaceutical composition further comprises a population of modified immune cells.

In certain embodiments, the method further comprises administering a pharmaceutical composition comprising a population of modified immune cells.

In certain embodiments, the modified immune cells have expression of synthetic receptors (e.g., CARs or TCRs) on the cell surface.

In certain embodiments, the immune cell is a T cell, a Natural Killer (NK) cell, a NKT cell, a B cell, a macrophage cell, an eosinophil or a neutrophil.

In certain embodiments, the immune cell is a T cell, selected from the group consisting of CD4+ T cell, CD8+ T cell, cytotoxic T cell, terminal effector T cell, memory T cell, naïve T cell, natural killer T cell, gamma-delta T cell, cytokine-induced killer (CIK) T cell, and tumor infiltrating lymphocyte.

In certain embodiments, the immune cell is autologous or allogeneic.

In certain embodiments, the cancer is a solid cancer selected from the group consisting of adrenal cancer, bone cancer, brain cancer, breast cancer, colorectal cancer, esophageal cancer, eye cancer, gastric cancer, head and neck cancer, kidney cancer, liver cancer, lung cancer, non-small cell lung cancer, bronchioloalveolar cell lung cancer, mesothelioma, head and neck cancer, squamous cell carcinoma, melanoma, oral cancer, ovarian cancer, cervical cancer, penile cancer, prostate cancer, pancreatic cancer, skin cancer, sarcoma, testicular cancer, thyroid cancer, uterine cancer, vaginal cancer.

In certain embodiments, the cancer is a hematologic malignancy selected from the group consisting of diffuse large B-cell lymphoma (DLBCL), extranodal NK/T-cell lymphoma, HHV8-associated primary effusion lymphoma, plasmablastic lymphoma, primary CNS lymphoma, primary mediastinal large B-cell lymphoma, T-cell/histiocyte-rich B-cell lymphoma, Hodgkin's lymphoma, non-Hodgkin's lymphoma, Waldenstrom's macroglobulinemia, multiple myeloma (MN).

In another aspect, the present disclosure provides a method of inducing proliferation of immune cells, prolonging the survival of immune cells, and/or increasing expression and/or secretion of immune stimulating cytokines from immune cells in an immune suppressive microenvironment, comprising contacting the immune suppressive microenvironment with the engineered cell provided herein.

In certain embodiments, the immune cell is a T cell, a Natural Killer (NK) cell, a NKT cell, a B cell, a macrophage cell, an eosinophil or a neutrophil.

In certain embodiments, the immune cell is a T cell, selected from the group consisting of CD4+ T cell, CD8+ T cell, cytotoxic T cell, terminal effector T cell, memory T cell, naïve T cell, natural killer T cell, gamma-delta T cell, cytokine-induced killer (CIK) T cell, and tumor infiltrating lymphocyte.

In certain embodiments, the immune cell is autologous or allogeneic.

In certain embodiments, the immune suppressive microenvironment is an immune suppressive tumor microenvironment.

In certain embodiments, the immune suppressive tumor microenvironment comprises a tumor and/or a tumor infiltrating immune cell expressing an immune inhibitory molecule.

In certain embodiments, the immune inhibitory molecule is selected from the group consisting of PD-1, TIM-3, TIGIT, LAG-3, A2AR, BTLA (CD272), CTLA-4 (CD152), IDO1, IDO2, TDO, NOX2, VISTA, SIGLEC7 (CD328), PVR(CD155) and SIGLEC9 (CD329), PD-L1, PD-L2, B7-H3 (CD276), B7-H4 (VTCN1), PVR(CD155), sialoglycoprotein, CD112, CD113, Galectin9, CD24, and CD47.

In certain embodiments, the immune inhibitory molecule is CTLA-4 and/or PD-L1.

In certain embodiments, the tumor comprises a cell expressing CTLA4-Ig and/or PD-L1.

In certain embodiments, the immune stimulating cytokines are one or more of TNF-α, IFN-β, IFN-γ, IL-1, IL-2, IL-4, IL-6, IL-8, IL-10, IL-12, IL-18 and granulocyte-macrophage colony stimulating factor.

In another aspect, the present disclosure provides a method of treating a disease or pathological condition in a subject in need thereof, comprising administering a therapeutically effective amount of the pharmaceutical composition provided herein.

In certain embodiments, the method provided herein further comprises administering a second agent.

In certain embodiments, the second therapy is a population of modified immune cells.

In certain embodiments, the second therapy is CAR-T therapy.

In certain embodiments, the disease comprises a cancer.

In another aspect, the present disclosure provides a method of selecting a CAR capable of activating dendritic cells, comprising:
(a) providing a non-human animal comprising an immune suppressive tumor microenvironment,
(b) administering a dendritic cell expressing a candidate CAR to the non-human animal,
(c) detecting a marker for the dendritic cell activation selected from improved infiltration to the immune suppressive tumor microenvironment, improved survival rate, and enhanced function in inducing activation of an immune cell when compared to a reference dendritic cell, and
(d) selecting the candidate CAR as a CAR capable of activating dendric cells.

In certain embodiments, the immune suppressive tumor microenvironment is clinically relevant.

In certain embodiments, the non-human animal comprises human fetal thymus and autologous human hematopoietic stem cells (e.g., human CD34+ hematopoietic stem cells).

In certain embodiments, the immune suppressive tumor microenvironment comprises a tumor and/or tumor infiltrating immune cells expressing an immune inhibitory molecule.

In certain embodiments, the immune inhibitory molecule is selected from the group consisting of PD-1, TIM-3, TIGIT, LAG-3, A2AR, BTLA (CD272), CTLA-4 (CD152), IDO1, IDO2, TDO, NOX2, VISTA, SIGLEC7 (CD328), PVR(CD155) and SIGLEC9 (CD329), PD-L1, PD-L2, B7-H3 (CD276), B7-H4 (VTCN1), PVR(CD155), HLA class I, sialoglycoprotein, CD112, CD113, Galectin9, CD24, and CD47.

In certain embodiments, the immune inhibitory molecule is CTLA-4 and/or PD-L1.

In certain embodiments, the tumor comprises a cell expressing CTLA4-Ig and/or PD-L1.

In certain embodiments, the immune cell is a T cell, a Natural Killer (NK) cell, a NKT cell, a B cell, a macrophage cell, an eosinophil or a neutrophil.

In certain embodiments, the immune cell is a T cell selected from the group consisting of CD4+ T cell, CD8+ T cell, cytotoxic T cell, terminal effector T cell, memory T cell, naïve T cell, natural killer T cell, gamma-delta T cell, cytokine-induced killer (CIK) T cell, and tumor infiltrating lymphocyte.

In certain embodiments, the immune cell is autologous or allogeneic.

In certain embodiments, the immune cell is a modified immune cell (e.g., CAR-T cells) or a native immune cell.

In certain embodiments, the modified immune cell (e.g., CAR-T cells) is administered in combination with the dendritic cell expressing the candidate CAR.

In certain embodiments, the non-human animal is a rodent, such as a rat or a mouse.

### BRIEF DESCRIPTION OF THE DRAWINGS

The accompanying drawings, which are incorporated herein, form part of the specification. Together with this written description, the drawings further serve to explain the principles of, and to enable a person skilled in the relevant art(s), to make and use the present disclosure.

FIG. 1 shows that CARDF enhanced the activity of DCs derived from THP-1 cells. FIG. 1A shows schematic diagram of various anti-CD19 CAR molecules. FIG. 1B shows that the CARDF expression on the surface of THP-1 cell line was determined by flow cytometry. CARDF was detected by its binding to protein L. FIG. 1C shows flow cytometric analysis of the differentiation efficiency of CARDF⁺ THP-1 cells into DCs. FIG. 1D shows the expression of co-stimulatory molecules CD80 and CD86 by Control-DCs and CARDF-DCs after they had been co-cultured with CD19′ H460 tumor cells for 2 days. FIG. 1E shows the proliferation of CD3⁺ primary T cells labelled with CFSE was analyzed by flow cytometry after being co-cultured with Control-DCs or CARDF-DCs for 3 days. DCs were activated by CD19+H460 tumor cells for 2 days as described in FIG. 1D. Histogram on the right showed Median fluorescence intensity (MFI) of CFSE in T cells. n=3. FIG. 1F shows that the expression of CD19 on the surface of H460 cells was analyzed by flow cytometry. FIG. 1G shows that the expression of anti-CD19 CAR on CAR-T cells was analyzed with protein L binding. n=3. FIG. 1H shows specific killing capability of CAR-T cells against CD19′ H460 tumor cells in the presence of Control-DCs or CARDF-DCs for 24 hours. n=3. FIGS. 1I and 1J show that the levels of IFN-γ (FIG. 1I) were assessed by ELISA and LDH (FIG. 1J) analyzed by CytoTox 96® Assay in the supernatant of the co-cultures of FIG. 1H. n=3. Data are presented as mean value±SD. Statistics: one-way ANOVA, Brown-Forsythe test with Tukey's multiple comparisons test. n.d., not detected; *P<0.05, **P<0.01, ***P<0.001, ****P<0.0001, ns indicates no significant.

FIG. 2 shows that CARDF-DCs derived from peripheral monocytes exhibit robust T cell activating activities in vitro. FIG. 2A shows that the expression of CARDF on the surface of DCs derived from monocytes (Mo-DCs) was analyzed by flow cytometry. Mock-DCs transduced with the empty-vector lentivirus were used as control. FIG. 2B shows that the expression of various DC-specific markers in Mo-DCs, Mock-DCs, and CARDF-DCs after being stimulated by LPS and TNF-α. n=3. FIG. 2C shows that the proliferation of CD3⁺ primary T cells, assessed by CellTrace-CFSE dilution, was analyzed after being co-cultured with Mock-DCs or CARDF-DCs for 3 days. DCs had been pre-exposed to EPHA2+A549 for 48 hours. Histogram on the right showed the MFI of CFSE of T cells. n=3. Statistics: one-way ANOVA, Brown-Forsythe test with Tukey's multiple comparisons test. FIG. 2D shows that the PD-L1 expression on A549-CP was analyzed by flow cytometry and CTLA4-Ig assessed by RT-qPCR. n=3. Statistics: unpaired two-tailed Student's t test. FIG. 2E shows that the surface expression of the activation markers in Mock-DCs and CARDF-DCs before and after co-culture with A549-CP for 48 hours. n=3. Statistics: unpaired two-tailed Student's t test. FIG. 2F shows that proliferation of CD3⁺ primary T cells, assessed by CellTrace-CFSE dilution, was analyzed after being co-cultured with Mock-DCs or CARDF-DCs for 4 days in the presence of A549-CP. Histogram on the right showed the MFI of CFSE of all T cells. n=3. Data are represented as mean value±SD. Statistics: Unpaired two-tailed Student's t test. *P<0.05, **P<0.01, ***P<0.001, ****P<0.0001.

FIG. 3 shows that CARDF-DCs activate the cytotoxicity of CAR-T cells against A549CP cells in vitro. FIG. 3A shows that the expression of CAR (scFv: anti-EphA2) on CAR-T cells. FIG. 3B shows that the expression of EphA2 on A549 and A549CP was analyzed by flow cytometry. FIG. 3C shows cytolytic capability of CAR-T cells against A549 and A549CP tumor cells in the presence of Mock-DCs or CARDF-DCs for 24 hours. n=3. FIG. 3D shows RT-qPCR analysis of IFN-γ, IL-2 and TNF-α expression in CAR-T cells from A549CP condition as in (C). n=3. FIG. 3E shows flow cytometric analysis of IFN-γ′ cells in CD8⁺ CAR-T cells from cultures in FIG. 3C. FIGS. 3F and 3G show that levels of IFN-γ (FIG. 3F) was assessed by ELISA and LDH (FIG. 3G) was analyzed by CytoTox 96® Assay in the supernatant collected from cultures in FIG. 3C. n=3. Data are presented as mean value±SD. n=3. Statistics: one-way ANOVA, Brown-Forsythe test with Tukey's multiple comparisons test. *P<0.05, **P<0.01, ***P<0.001, ****P<0.0001, ns indicates no significant.

FIG. 4 shows that CARDF-DCs activate Car-T cells to eliminate solid lung tumors with TIME. FIG. 4A shows experimental design to treat A549WT and A549CP lung tumors formed in NSG mice with CARDF-DCs and Car-T cells. FIG. 4B shows that expression of immune suppressive genes in A549WT and A549CP tumors assessed by RT-qPCR. Data are presented as mean value±SD. n=3. Statistics: unpaired two-tailed Student's t test. FIG. 4C shows photographs of tumors recovered on day 17 after treatments described in FIG. 4A. Left: A549WT tumors, right: A549CP tumors. FIG. 4D shows weight of tumors shown in (FIG. 4C). Data are presented as mean value±SD. n=5. Statistics: one-way ANOVA, Brown-Forsythe test with Tukey's multiple comparisons test. FIG. 4E shows gene expression in recovered A549CP tumors shown in FIG. 4C by RT-qPCR. Data are presented as mean value±SD. n=5. FIG. 4F shows that percentages of total T cells, CD8+ T cells, dendritic cells, CD80⁺ dendritic cells, CD86⁺ dendritic cells in spleen were analyzed by flow cytometry. Data are presented as mean value±SD. n=5. *P<0.05, **P<0.01, * **P<0.001, ****P<0.0001, ns indicates no significant.

FIG. 5 shows that CARDF-DCs promote CAR-T cell mediated regression of lung tumors formed in Hu-mice. FIG. 5A shows experimental design of treating A549 lung tumors formed in Hu-mice. FIG. 5B shows the expression of genes in lung tumors from NSG mice and Hu-mice after CAR-T cell treatment assessed by RT-qPCR. Data are presented as mean value±SD. n=3. Statistics: unpaired two-tailed Student's t test. FIG. 5C shows volumes of tumors after various treatments. Data are presented as mean value±SD. Statistics: two-way ANOVA followed by Tukey's multiple comparisons test. FIGS. 5D and 5F show photograph of tumors recovered on day 16 after inoculation (FIG. 5D) and tumors weight (FIG. 5E). Treatment course is indicated in FIG. 5A. Data are presented as mean value±SD. Statistics: one-way ANOVA, Brown-Forsythe test with Tukey's multiple comparisons test. Normal T: n=4; CAR-T: n=6; Mock-DCs: n=6; CARDF-DCs: n=6. *P<0.05, **P<0.01, ***P<0.001, ****P<0.0001.

FIG. 6 shows that CARDF-DCs reverse TIME of lung tumors formed in Hu-mice towards a pro-inflammatory state. FIG. 6A shows that percentage of IFN-γ⁺ T cells in splenic CD3+ T cells was analyzed by flow cytometry. Data are presented as mean value±SD. n=2 for Normal T; n=3 for CAR-T, Mock-DCs, CARDF-DCs. FIG. 6B shows that PD-1 and TIM-3+ T cells in spleen were analyzed by flow cytometry. FIG. 6C shows that MFI of CD86 and MHC-II expressed by dendritic cells from spleen were analyzed by flow cytometry. Data are presented as mean value±SD. n=2 for Normal T; n=3 for CAR-T, Mock-DCs, CARDF-DCs. FIG. 6D shows that the expression of TNF-α, IL-2, CD86 and IL-12B in dissected lung tumors was assessed by RT-qPCR. Normalization was indicated in the figure. Data are presented as mean value±SD. n=2 for Normal T; n=3 for CAR-T, Mock-DCs, CARDF-DCs. FIG. 6E shows percentage of PD-1⁺ TIM-3+ T cells in splenic CD3+ T cells as indicated in FIG. 6B. Data are presented as mean value±SD. n=2 for Normal T; n=3 for CAR-T, Mock-DCs, CARDF-DCs. FIGS. 6F and 6G show that the expression of PD-1, TIM-3, TGF-3 (FIG. 6F) or CD206 and CD163 (FIG. 6G) in dissected lung tumors was assessed by RT-qPCR. Normalization was indicated in the figure. Data are presented as mean value±SD. n=2 for Normal T; n=3 for CAR-T, Mock-DCs, CARDF-DCs. *P<0.05, **P<0.01, * **P<0.001, ****P<0.0001.

FIG. 7 shows that CARDF-DCs can resist TIME of distinct lung tumors to activate CAR-T cells. FIG. 7A shows that PD-L1 expression on A549 and H460 tumor cell line was analyzed by flow cytometry. FIG. 7B shows that relative gene expression in A549 tumors and H460 tumors formed in Hu-mice was assessed by RT-qPCR. Data are presented as mean value±SD. n=3. FIG. 7C shows that EphA2 expression on H460 lung tumor cells was detected by flow cytometry. FIG. 7D shows schematic design of CAR-T and DC combined therapy of H460 tumors formed in Hu-mice. FIG. 7E shows CAR (scFv: anti-EphA2) expression on the surface of DCs and T cells derived from Hu-mice generated with the same fetal tissues as the tumor-harboring Hu-mice. FIG. 7F shows growth curves of tumors after various treatments. Data are presented as mean value±SD, n=6. Statistics: two-way ANOVA followed by Tukey's multiple comparisons test. FIG. 7G shows that MFI of CD80 and CD86 on DCs infiltrating in tumors were analyzed by flow cytometry. Data are presented as mean value±SD, n=3. *P<0.05, **P<0.01, ***P<0.001, ****P<0.0001.

FIG. 8A and FIG. 8B show screening of CARs that activate DCs derived from THP-1 cells. FIG. 8A shows that the expression of CAR (scFv: anti-CD19 and 2^{nd} generation T cell activation domain, or TLR4 activating domain, or TNFR2 activating domain) on surface of THP-1 cells was determined by binding to protein L. FIG. 8B shows that the expression of co-stimulatory molecules CD80 and CD86 on DCs after co-culture with H460-CD19 tumor cells for 2 days.

FIG. 9A and FIG. 9B show schematic diagram of anti-EphA2 CAR molecules.

FIG. 9A shows diagram of the lentiviral vectors containing anti-EphA2 CAR construct for DCs (CARDF). FIG. 9B shows diagram of the lentiviral vectors containing anti-EphA2 CAR construct for T cells (2^{nd} generation, B).

FIG. 10A shows antibodies used in the present disclosure.

FIG. 10B shows primer sequences for RT-qPCR used in the present disclosure.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definition

The following definitions are provided to assist the reader. Unless otherwise defined, all terms of art, notations and other scientific or medical terms or terminology used herein are intended to have the meanings commonly understood by those of skill in the art. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps. Terms such as “consisting essentially of” and “consists essentially of” have the meaning attributed in United States Patent law; they allow for the inclusion of additional ingredients or steps that do not materially affect the basic and novel characteristics of the claimed invention. The terms “consists of” and “consisting of” have the meaning ascribed to them in United States Patent law; namely that these terms are close ended.

In all occurrences in this application where there are a series of recited numerical values, it is to be understood that any of the recited numerical values may be the upper limit or lower limit of a numerical range. It is to be further understood that the invention encompasses all such numerical ranges, i.e., a range having a combination of an upper numerical limit and a lower numerical limit, wherein the numerical value for each of the upper limit and the lower limit can be any numerical value recited herein. Ranges provided herein are understood to include all values within the range. For example, 1-10 is understood to include all of the values 1, 2, 3, 4, 5, 6, 7, 8, 9, and 10, and fractional values as appropriate. Similarly, ranges delimited by “at least” are understood to include the lower value provided and all higher numbers.

As used herein, “about” is understood to include within three standard deviations of the mean or within standard ranges of tolerance in the specific art. In certain embodiments, about is understood as a variation of no more than 0.5.

As used herein, the term “CAR”, which can be used interchangeably with the term “chimeric antigen receptor” refers to an engineered receptor or a synthetic receptor or polynucleotide encoding thereof. The engineered receptor or a synthetic receptor comprises an extracellular domain that comprises an antigen binding domain, a transmembrane domain, and/or an intracellular signaling domain, optionally a signal peptide, which are joined one another or operably linked to each other. The most common CARs are, for example, a single-chain variable fragment (scFv) derived from a monoclonal antibody fused to CD3-zeta transmembrane and endodomain. Such CARs result in the transmission of a zeta signal in response to specific binding of scFv to its target. Methods of preparing CARs are publicly available (see, e.g., Grupp et al., N Engl J Med., 368:1509-1518, 2013; Park et al., Trends Biotechnol., 29:550-557, 2011; Haso et al., (2013) Blood, 121, 1165-1174; Han et al., J. Hematol Oncol. 6:47, 2013; WO2012/079000; U.S. Pub. 2012/0213783; and WO2013/059593, each of which is incorporated by reference herein in its entirety).

The term “chimeric antigen receptor T cell”, used interchangeably with the term “CAR-T cell”, refers to a T cell or population thereof that has been engineered through biological methods (e.g., genetic engineering) to express a CAR on the T cell surface. CAR-T cells can be T helper CD4+ and/or T effector CD8+ cells. CAR-T can identify surface antigens and initiate immune response.

“Antigen” refers to a molecule that provokes an immune response. This immune response may be either humoral, or cell-mediated response, or both. The skilled artisan will understand that any macromolecule, including virtually all proteins or peptides, can serve as an antigen. It is readily apparent that the present disclosure includes therapeutic antibodies acting as antigen eliciting immune response.

“Antibody” refers to a polypeptide of the immunoglobulin (Ig) family that binds with an antigen. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR) (light chain CDRs including LCDR1, LCDR2, and LCDR3, heavy chain CDRs including HCDR1, HCDR2, HCDR3), interspersed with regions that are more conserved, termed framework regions (FR). CDR boundaries for the antibodies disclosed herein may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. Dec. 5; 186(3):651-63 (1985); Chothia, C. and Lesk, A. M, J. Mol. Biol., 196,901 (1987); Chothia, C. et al., Nature. Dec. 21-28; 342(6252):877-83 (1989); Kabat E. A. et al., National Institutes of Health, Bethesda, Md. (1991); Marie-Paule Lefranc et al, Developmental and Comparative Immunology, 27: 55-77 (2003); Marie-Paule Lefranc et al, Immunome Research, 1(3), (2005); Marie-Paule Lefranc, Molecular Biology of B cells (second edition), chapter 26, 481-514, (2015)). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen.

“Antigen-binding domain” as used herein refers to an antibody fragment formed from a portion of an intact antibody comprising one or more CDRs, or any other antibody fragment that can bind to an antigen but does not comprise an intact native antibody structure. Examples of antigen-binding domain include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), single-chain Fv-Fc antibody (scFv-Fc), an scFv dimer (bivalent diabody), a bispecific antibody, a multispecific antibody, a camelized single domain antibody, a nanobody, a domain antibody, and a bivalent domain antibody. An antigen-binding domain is capable of binding to the same antigen to which the parent antibody binds.

“Autologous” cells refer to any cells derived from the same subject into which they are later to be re-introduced.

“Allogeneic” cells refer to any cells derived from a different subject of the same species.

“Effector cells” used in the context of immune cells refers to cells that can be activated to carry out effector functions in response to stimulation. Effector cells may include, without limitation, NK cells, cytotoxic T cells and helper T cells.

“Effective amount” or “therapeutically effective amount” refers to an amount of a cell, composition, formulation or any material as described here effective to achieve a desirable biological result. Such results may include, without limitation, elimination of B cells expressing a specific BCR and the antibodies produced therefrom.

Percentage of “identity” or “sequence identity” in the context of polypeptide or polynucleotide is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “conservative substitution”, as used herein with reference to amino acid sequence refers to replacing an amino acid residue with a different amino acid residue having a side chain with similar physiochemical properties. For example, conservative substitutions can be made among amino acid residues with hydrophobic side chains (e.g. Met, Ala, Val, Leu, and Ile), among residues with neutral hydrophilic side chains (e.g. Cys, Ser, Thr, Asn and Gln), among residues with acidic side chains (e.g. Asp, Glu), among amino acids with basic side chains (e.g. His, Lys, and Arg), or among residues with aromatic side chains (e.g. Trp, Tyr, and Phe). As known in the art, conservative substitution usually does not cause significant change in the protein conformational structure, and therefore could retain the biological activity of a protein.

The term “functional forms” as used herein, refers to different forms (such as variants, fragments, fusions, derivatives and mimetics) of the parent molecule, which, despite of having difference in amino acid sequences or in chemical structures, still retains substantial biological activity of the parent molecule. The expression “retain substantial biological activity”, as used herein, means exhibiting at least part of (for example, no less than about 20%, 30%, 40%, 50%, 60%, 70%, 80%, or 90%) or all of the biological activity of the parent molecule. A functional form of a parent polypeptide may include both naturally-occurring variant forms and non-naturally occurring forms such as those obtained by recombinant methods or chemical synthesis. The functional forms may contain non-natural amino acid residues.

As used herein, the term “operably linked” refers to a functional relationship between two or more polynucleotide sequences. In the context of a polynucleotide encoding a fusion protein, such as a polypeptide chain of a CAR of the disclosure, the term means that the two or more polynucleotide sequences are joined such that the amino acid sequences encoded by these segments remain in-frame. In the context of transcriptional or translational regulation, the term refers to the functional relationship of a regulatory sequence to a coding sequence, for example, a promoter in the correct location and orientation to the coding sequence so as to modulate the transcription.

As used herein, the term “polynucleotide” or “nucleic acid” refers to a chain of nucleotides. They also refer to synthetic and/or non-naturally occurring nucleic acid molecules (e.g., comprising nucleotide analogues or modified backbone residues or linkages). The terms also refer to deoxyribonucleotide or ribonucleotide oligonucleotides in either single-stranded or double-stranded form. The terms encompass nucleic acids containing analogues of natural nucleotides. The terms also encompass nucleic acid-like structures with synthetic backbones. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “polypeptide”, “peptide” and “protein” are used interchangeably herein to refer to a polymer of amino acid residues. The terms also apply to amino acid polymers in which one or more amino acid residue is an artificial chemical mimetic of a corresponding naturally-occurring amino acid, as well as to naturally-occurring amino acid polymers and non-naturally occurring amino acid polymers. In certain embodiments, the polypeptides include natural peptides, recombinant peptides, synthetic peptides, or a combination thereof.

As used herein, the term “single-chain variable fragment” used interchangeably with the term “scFv” refers to an engineered antibody consisting of a light chain variable region and a heavy chain variable region connected to one another directly or via a peptide linker sequence (Huston J S et al. Proc Natl Acad Sci USA, 85:5879 (1988)).

As used herein, the term “TCR”, which can be used interchangeably with the term “T cell receptor” or the term “TCR complex” refers to a natural (or endogenous) TCR or an engineered TCR. TCR refers to a protein complex on the surface of T cells that is responsible for recognizing fragments of antigen as peptides bound to MHC molecules

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the fusion polypeptide, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pProl8, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

The phrase “host cell” as used herein refers to a cell into which an exogenous polynucleotide and/or a vector has been introduced.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “subject” or “individual” or “animal” or “patient” as used herein refers to human or non-human animal, including a mammal or a primate, in need of diagnosis, prognosis, amelioration, prevention and/or treatment of a disease or disorder. Mammalian subjects include humans, domestic animals, farm animals, and zoo, sports, or pet animals such as dogs, cats, guinea pigs, rabbits, rats, mice, horses, swine, cows, bears, and so on.

The term “treating”, or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

### Dendritic Cell (DC)-Activating Chimeric Antigen Receptor (CAR)

The present disclosure provides a polynucleotide (e.g., DNA or RNA) encoding a chimeric antigen receptor (CAR) that is capable of activating dendritic cells (DCs) in an immune suppressive tumor microenvironment or tumor immune suppressive microenvironment (TIME). The term “immune suppressive tumor microenvironment” and the term “TIME” can be used interchangeably, and refers to a microenvironment having, for example, tumor cells, tumor infiltrating immune cells, tumor associated fibroblasts, endothelial cells, and various chemotactic and inflammatory or immune stimulating cytokines, which, together with a dense extracellular matrix, capable of suppressing tumor immune surveillance and immunotherapy (F. R. Balkwill et al., The tumor microenvironment at a glance. J. Cell Sci. 125, 5591-5596 (2012); M. Binnewies et al., Understanding the tumor immune microenvironment (TIME) for effective therapy. Nat Med. 24, 541-550 (2018); M A.-M Alireza Labani-Motlagh et al., The Tumor Microenvironment: A Milieu Hindering and Obstructing Antitumor Immune Responses. Front. Immunol. 11, 940 (2020) and L. Hui et al., Tumor microenvironment: Sanctuary of the devil. Cancer Lett. 368, 7-13 (2015)).

In certain embodiments, the immune suppressive tumor microenvironment or TIME comprises a solid tumor and/or tumor infiltrating immune cells expressing an immune inhibitory molecule. The immune inhibitory molecule can be selected from the group consisting of PD-1, TIM3, TIGIT, LAG3, A2AR, BTLA (CD272), CTLA-4 (CD152), IDO1, IDO2, TDO, NOX2, VISTA, SIGLEC7 (CD328), PVR(CD155) and SIGLEC9 (CD329), PD-L1, PD-L2, B7-H3 (CD276), B7-H4 (VTCN1), PVR(CD155), HLA class I, sialoglycoprotein, CD112, CD113, Galectin9, CD24, and CD47. In certain embodiments, the immune inhibitory molecule is CTLA-4 and/or PD-L1. As used herein, the term “expressing” or “express” with respect to an immune inhibitory molecule, refers to expressing an immune inhibitory molecule at a level that is at least 2 folds, at least 3 folds, at least 4 folds, at least 5 folds, at least 6 folds, at least 7 folds, at least 8 folds, at least 9 folds, at least 10 folds, at least 15 folds, at least 20 folds, at least 25 folds, at least 30 folds, at least 35 folds, at least 40 folds, at least 60 folds, at least 80 folds, at least 100 folds, at least 120 folds, at least 150 folds, at least 200 folds, at least 300 folds, at least 400 folds, at least 500 folds, at least 600 folds, at least 700 folds, at least 800 folds, at least 900 folds or at least 1000 folds higher than a reference level. The term “reference level” with respect to the expression of an immune inhibitory molecule refers to an expression level of the immune inhibitory molecule in a tumor formed by wild-type tumor cells (e.g., wild-type A549 cells) in an immune-deficient animal model (e.g., NSG mouse).

“CTLA-4” is short for Cytotoxic T-Lymphocyte-Associated protein 4 and is also known as CD152, and more detailed description can be found in, for example, Kolar et al., (Jan. 1, 2009) CTLA-4 (CD152) controls homeostasis and suppressive capacity of 10 regulatory T cells in mice. Arthritis Rheum. 60 (1): 123-32. “PD-L1” is short for programmed death-ligand 1 and is also known as cluster of differentiation 274 (CD274) or B7 homolog 1 (B7-H1), and more detailed description can be found in, for example, Dong H et al., B7-H1, a third member of the B7 family, co-stimulates T-cell proliferation and interleukin-10 secretion. Nature Medicine. 5 (12): 1365-9, 1999.

CTLA-4 and PD-L1 are critical immune inhibitory molecules in maintaining peripheral tolerance by restraining T cell activity. CTLA-4 binds to CD80 and CD86 with higher affinity than CD28, which are the primary co-stimulation pathways for activating T cells. PD-L1 binds to PD-1 that is expressed on T cell surface and inhibits T cell activity. PD-L1 plays a central role in maintaining T cell anergy and preventing autoimmunity (Walker L S K et al., The enemy within: keeping self-reactive T cells at bay in the periphery. Nat Rev Immunol. 2002; 2:11-9; Fife B T et al., Control of peripheral T-cell tolerance and autoimmunity via the CTLA-4 and PD-1 pathways. Immunological Reviews. 2008; 224:166-182; and Keir M E et al., PD-1 and Its Ligands in Tolerance and Immunity. Annual Review of Immunology. 2008; 26:677-704.).

In certain embodiments, the tumor within TIME comprises a cell expressing CTLA-4-immunoglobulin fusion protein (CTLA4-Ig) and/or PD-L1. CTLA4-Ig has been developed to inhibit T cell-mediated immune responses (Walker L S K et al., The enemy within: keeping self-reactive T cells at bay in the periphery. Nat Rev Immunol. 2002; 2:11-19.). As used herein, the term “expressing” or “express” with respect to CTLA4-Ig, refers to expressing CTLA4-Ig at a level that is at least 2 folds, at least 3 folds, at least 4 folds, at least 5 folds, at least 6 folds, at least 7 folds, at least 8 folds, at least 9 folds, at least 10 folds, at least 15 folds, at least 20 folds, at least 25 folds, at least 30 folds, at least 35 folds, at least 40 folds, at least 60 folds, at least 80 folds, at least 100 folds, at least 120 folds, at least 150 folds, at least 200 folds, at least 300 folds, at least 400 folds, at least 500 folds, at least 600 folds, at least 700 folds, at least 800 folds, at least 900 folds or at least 1000 folds higher than a reference level. The term “reference level” with respect the expression of CTLA4-Ig refers to an expression level of the CTLA4-Ig in a wild-type tumor cell (e.g., wild-type A549 cells). As used herein, the term “expressing” or “express” with respect to PD-L1, refers to expressing PD-L1 at a level that is at least 2 folds, at least 3 folds, at least 4 folds, at least 5 folds, at least 6 folds, at least 7 folds, at least 8 folds, at least 9 folds, at least 10 folds, at least 15 folds, at least 20 folds, at least 25 folds, at least 30 folds, at least 35 folds, at least 40 folds, at least 60 folds, at least 80 folds, at least 100 folds, at least 120 folds, at least 150 folds, at least 200 folds, at least 300 folds, at least 400 folds, at least 500 folds, at least 600 folds, at least 700 folds, at least 800 folds, at least 900 folds or at least 1000 folds higher than a reference level. The term “reference level” with respect the expression of PD-L1 refers to an expression level of the PD-L1 in a wild-type tumor cell (e.g., wild-type A549 cells).

In certain embodiments, the CTLA-4-Ig comprises an amino acid sequence set forth in SEQ ID NO: 8 or a sequence having at least 75%, 80%, 85%, 90%, 95%, or 99% identity thereto while retaining substantial biological activity of SEQ ID NO: 8, or a sequence having 1, 2, 3, 4, 5, 6, 7, 8, 9 or 10 conservative substitutions thereof, or any functional forms thereof. In certain embodiments, the PD-L1 comprises an amino acid sequence set forth in SEQ ID NO: 9 or a sequence having at least 75%, 80%, 85%, 90%, 95%, or 99% identity thereto while retaining substantial biological activity of SEQ ID NO: 9, or a sequence having 1, 2, 3, 4, 5, 6, 7, 8, 9 or 10 conservative substitutions thereof, or any functional forms thereof.

In certain embodiments, the immune suppressive tumor microenvironment comprises a tumor that has poor responsiveness to monotherapy of adoptive cell therapy (e.g., CAR-T monotherapy). As used herein and throughout the specification, the term “poor responsiveness” refers to absence or reduced of responsiveness, which can be detected by a comparable (for example, less than 20%, less than 15%, less than 10%, less than 5%, less than 4%, less than 3% or less than 2% better therapeutical effect, and preferably less than 10% better therapeutical effect) therapeutical effect of a therapy (e.g., CAR-T therapy) as compared to a control treatment that is known to have no therapeutical effect.

Dendritic cells are professional antigen-presenting cells that can prime naïve T cells and reactivate memory responses. In cancer, dendritic cells can activate T cells (e.g., cytotoxic CD8+ T cells) through cross-presentation of tumor associated antigens (TAAs) or neoantigens to elicit a stronger anti-tumor response. The activation of a DC can be assayed by measuring various parameters, including, without limitation, the activation status of DC and/or the activation status of immune cells (e.g., T cells, microphages), which can be indicated by the expression level of DC activating markers (such as CD80, CD86 and MHC-II, CD83, CD54, CMRF-44, CMRF-56, type III INF, IL-12, CXCL9/10, IRF8)), the survival and/or cytotoxicity of the immune cells (e.g., T cells), the expression (and/or secretion) of immune stimulating cytokines (e.g., TNF-α, IFN-β, IFN-γ, IL-1, IL-2, IL-4, IL-6, IL-8, IL-10, IL-12, IL-18 and granulocyte-macrophage colony stimulating factor) from the immune cells (e.g., T cells), the expression level of immune inhibitory molecules (e.g., PD-1, TIM-3, TIGIT, LAG3, A2AR, BTLA (CD272), CTLA-4 (CD152), IDO1, IDO2, TDO, KIR, NOX2, VISTA, SIGLEC7 (CD328), PVR(CD155), and SIGLEC9 (CD329)) from the immune cells (e.g., T cells), and/or the expression level of markers for anti-inflammatory macrophages (e.g., M2 macrophages), such as CD206 and CD163.

In certain embodiments, the activation of dendritic cells comprises increased expression level of DC activating markers (such as CD80, CD86 and/or MHC-II, CD83, CD54, CMRF-44, CMRF-56, type III INF, IL-12, CXCL9/10, IRF8), increased survival of the immune cells (e.g., T cells (such as CD8+ T cells), DCs), increased expression (and/or secretion) of immune stimulating cytokines (e.g., TNF-α, IFN-β, IFN-γ, IL-1, IL-2, IL-4, IL-6, IL-8, IL-10, IL-12, IL-18 and/or granulocyte-macrophage colony stimulating factor) from the immune cells (e.g., T cells), decreased expression of immune inhibitory molecules (e.g., PD-1, TIM-3, TIGIT, LAG3, A2AR, BTLA (CD272), CTLA-4 (CD152), IDO1, IDO2, TDO, KIR, NOX2, VISTA, SIGLEC7 (CD328), PVR(CD155), and SIGLEC9 (CD329)) from the immune cells (e.g., T cells), and/or decreased expression level of markers (such as CD206 and CD163) for anti-inflammatory macrophages (e.g., M2 macrophages), when compared to a reference status (e.g., inactivated status) of dendritic cells.

In certain embodiments, the DC-activating CAR provided herein comprises: (1) an extracellular antigen-binding domain, (2) a transmembrane domain and (3) an intracellular signaling domain.

(1) Extracellular Antigen-Binding Domain

In some embodiments, the antigen binding domain comprises a human or humanized antibody or an antibody fragment thereof. The term “human antibody” refers to an antibody where the whole molecule is of human origin or consists of an amino acid sequence identical to a human form of the antibody or immunoglobulin. The term “humanized antibody” refers to an antibody which contains sequence (e.g., CDR sequences) derived from non-human immunoglobulin. Human or humanized antibodies or fragments thereof may be prepared in a variety of ways, for example through recombinant methodologies or through immunization with an antigen of interest of a mouse that is genetically modified to express antibodies derived from human heavy and/or light chain-encoding genes.

In some embodiments, the extracellular antigen-binding domain of the CAR provided herein comprises a single-chain variable fragment (scFv), a Fv, a Fab, a (Fab)2, an scFv, a nanobody, a non-covalently or covalently linked ligand/receptor domain or any alternative scaffold known in the art to function as antigen binding domain. In some embodiments, the extracellular antigen-binding domain of the CAR provided herein is a scFv. The scFv can be specific to a tumor surface marker, for example a solid tumor surface marker. In certain embodiments, the tumor surface marker is selected from the group consisting of: EphA2, CD19, CD70, CD117, CD133, CD147, CD171, DLL3, EGFRvIII, VGFR2, Mesothelin, ganglioside GD2, FAP (fibroblast activating protein), FBP (folate binding protein), LMP1, Lewis Y, Claudin 18.2, IL13Rα2, HER2, MDC1, PMSA (prostate membrane specific antigen), ROR1, ROR2, B7-H3, CAIX, CD133, CD171, CEA, GPC3, MUC1, MUC16, MAGE-A1, MAGE-A4, TROP2, EpCAM, NKG2D, other proteins found to be more highly enriched on the surface of tumor cells than critical normal tissues, and combination thereof. The extracellular antigen-binding domain can also be specific to non-tumor markers for diseases that can benefit from converting TIME towards a pro-inflammatory state, for example, markers for infectious diseases.

In some embodiments, the scFv is specific for EphA2. In certain embodiments, the scFv comprises a peptide linker of at least 0, 1, 2, 3, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In certain embodiments, the peptide linker comprises an amino acid sequence comprising SEQ ID NO: 57 (GGGGSGGGGSGGGGS).

In some embodiments, the scFv comprises a variable heavy (VH) and variable light (VL) region. In some embodiments, the VH comprises a heavy chain CDR1 (HCDR1) having a sequence set forth in SEQ ID NO: 10, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof, a CDR2 having a sequence set forth in SEQ ID NO: 11, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof; and a CDR3 having a sequence set forth in SEQ ID NO: 12, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof. In some embodiments, the VL region comprises a light chain CDR1 (LCDR1) having a sequence set forth in SEQ ID NO: 13, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof, a CDR2 having a sequence set forth in SEQ ID NO: 14, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof, and a CDR3 having a sequence set forth in SEQ ID NO: 15, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof.

In certain embodiments, the scFv comprises 1) a VH comprising a HCDR1 comprising a sequence set forth in SEQ ID NO: 10, a HCDR2 comprising a sequence set forth in SEQ ID NO: 11, a HCDR3 comprising a sequence set forth in SEQ ID NO: 12; and 2) a VL comprising a LCDR1 comprising a sequence set forth in SEQ ID NO: 13, a LCDR2 comprising a sequence set forth in SEQ ID NO: 14, a LCDR3 comprising a sequence set forth in SEQ ID NO: 15.

In some embodiments, the scFv comprises a VH and a VL. In certain embodiments, the VH comprises an amino acid sequence set forth in SEQ ID NO: 16, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof. In certain embodiments, the VL comprises an amino acid sequence set forth in SEQ ID NO: 17, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof. In some embodiments, the scFv comprises a VH comprising a sequence set forth in SEQ ID NO: 16, and a VL comprising a sequence set forth in SEQ ID NO: 17.

In some embodiments, the scFv comprises an amino acid sequence set forth in SEQ ID NO: 18.

The present disclosure successfully validated that CAR-DCs expressing EphA2-specific scFv can significantly reduce lung tumor volume in an immune suppressive environment. However, it should not be understood that the CAR-DCs provided herein can only be used to treat lung cancers. The skilled person in the art will appreciate that an appropriate extracellular antigen-binding domain specific for any disease marker may be selected to construct a CAR provided herein, depending on the disease of interest, in view of the existing knowledge of the identified markers for various diseases, such as cancer, infectious diseases, or immune diseases. The various disease markers include but not limited to those as described above.

(2) Transmembrane Domain

The transmembrane domain of the CAR described herein may be derived from any membrane-bound or transmembrane protein including, but are not limited to, BAFFR, BLAME (SLAMF8), CD2, CD3 epsilon, CD4, CD5, CD8, CD9, CD11a (CD18, ITGAL, LFA-1), CD11b, CD11c, CD11d, CD16, CD19, CD22, CD27, CD28, CD29, CD33, CD37, CD40, CD45, CD49a, CD49d, CD49f, CD64, CD80, CD84, CD86, CD96 (Tactile), CD100 (SEMA4D), CD103, CD134, CD137 (4-1BB), CD150 (IPO-3, SLAMF1, SLAM), CD154, CD160 (BY55), CD162 (SELPLG), CD226 (DNAM1), CD229 (Ly9), CD244 (2B4, SLAMF4), CD278 (ICOS), CEACAM1, CRT AM, GITR, HYEM (LIGHTR), IA4, IL2R beta, IL2R gamma, IL7R a, ITGA1, ITGA4, ITGA6, ITGAD, ITGAE, ITGAM, ITGAX, ITGB1, ITGB2, ITGB7, KIR, LTBR, OX40, NKG2C, NKG2D, NKp30, NKp44, NKp46, NKp80 (KLRF1), PAG/Cbp, PSGL1, SLAMF6 (NTB-A, Ly108), SLAMF7, an alpha, beta or zeta chain of a T-cell receptor, TNFR2, VLA1, and VLA-6.

In one embodiment, the CAR described herein comprises a transmembrane domain of CD8 alpha. In certain embodiments, the transmembrane domain of CD8 alpha has a sequence of SEQ ID NO: 6, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof.

In certain embodiments, the transmembrane domain of the CAR described herein is synthetic, e.g., comprising predominantly hydrophobic residues such as leucine and valine. In certain embodiment, the transmembrane domain of the CAR described herein is modified or designed to avoid binding to the transmembrane domains of the same or different surface membrane proteins in order to minimize interactions with other members of the receptor complex.

In some embodiments, the CAR described herein further comprises a hinge region, which forms the linkage between the extracellular domain and transmembrane domain of the CAR. The hinge and/or transmembrane domain provides cell surface presentation of the extracellular antigen-binding domain of the CAR.

The hinge region may be derived from any membrane-bound or transmembrane protein including, but are not limited to, BAFFR, BLAME (SLAMF8), CD2, CD3 epsilon, CD4, CD5, CD8, CD9, CD11a (CD18, ITGAL, LFA-1), CD11b, CD11c, CD11d, CD16, CD19, CD22, CD27, CD28, CD29, CD33, CD37, CD40, CD45, CD49a, CD49d, CD49f, CD64, CD80, CD84, CD86, CD96 (Tactile), CD100 (SEMA4D), CD103, CD134, CD137 (4-1BB), CD150 (IPO-3, SLAMF1, SLAM), CD154, CD160 (BY55), CD162 (SELPLG), CD226 (DNAM1), CD229 (Ly9), CD244 (2B4, SLAMF4), CD278 (ICOS), CEACAM1, CRT AM, GITR, HYEM (LIGHTR), IA4, IL2R beta, IL2R gamma, IL7Ra, ITGA1, ITGA4, ITGA6, ITGAD, ITGAE, ITGAM, ITGAX, ITGB1, ITGB2, ITGB7, KIR, LTBR, OX40, NKG2C, NKG2D, NKp30, NKp44, NKp46, NKp80 (KLRF1), PAG/Cbp, PSGL1, SLAMF6 (NTB-A, Ly108), SLAMF7, an alpha, beta or zeta chain of a T-cell receptor, TNFR2, VLA1, and VLA-6.

In some embodiments, the hinge region comprises a hinge region of CD8 alpha, a hinge region of human immunoglobulin (Ig), or a glycine-serine rich sequence.

In some embodiments, the CAR comprises a hinge region of CD8 alpha. In certain embodiments, the hinge region has a sequence of SEQ ID NO: 7, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof.

(3) Intracellular Signaling Domain

The intracellular signaling domain of the CAR described herein is responsible for activation of at least one of the normal effector functions of the immune cell (e.g., dendritic cell) in which the CAR has been placed in. The term “effector function” used in the context of an immune cell refers to a specialized function of the cell, for example, the phagocytic activity, cytolytic activity or helper activity. In certain embodiments, the intracellular signaling domain of the CAR described herein is capable of activating (including maturation) dendritic cells in an immune suppressive tumor microenvironment. Activation of DCs can be induced by many cell surface receptors, such as TLR4 (A. Iwasaki et al., Toll-like receptor control of the adaptive immune responses. Nat. Immunol. 5, 987-995 (2004).), TNFR (L. M Sedger et al., From mediators of cell death and inflammation to therapeutic giants—past, present and future. Cytokine Growth Factor Rev. 25, 453-472 (2014).), IFNγR (M. Z. Jianping Pan et al., Interferon-γ is an autocrine mediator for dendritic cell maturation. Immunol. Lett. 94, 141-151 (2004).), Dectin-1 (T. S. Helen S. et al., Differential utilization of CARD9 by Dectin-1 in macrophages and dendritic cells. J Immunol. 182, 1146-1154 (2009)) and FcγR (M. Guilliams et al., The function of Fcγ receptors in dendritic cells and macrophages. Nat. Rev. Immunol. 14, 94-108 (2014), T. H. Flinsenberg, Fc receptor antigen targeting potentiates cross-presentation by human blood and lymphoid tissue BDCA-3 dendritic cells. Blood 120, 26 (2012).) in response to various stimuli. These DC activating receptors have one or more immune-receptor tyrosine-based activation motif (ITAM) in their cytoplasmic domains, which triggers activating signal cascades to activate DCs. As used herein, the term “cytoplasmic domain” refers to a fully length domain of a protein residing inside cytoplasm, or any fragment thereof, for example, a fragment having a length that is at least 1%, at least 2%, at least 3%, at least 4%, at least 5%, at least 10%, at least 20%, at least 30%, at least 40%, at least 50%, at least 60%, at least 70%, at least 80%, at least 90%, or at least 95% of the full-length domain.

The intracellular signaling domain of the CAR described herein may comprise a cytoplasmic domain of a dendritic cell activating receptor selected from the group consisting of RIG-1, NLRP10, DEC-205, BDCA-2, CD86, 4-1BBL, OX40L, CD40, IFNAR, TLR4, TNFR (e.g., TNFR2), IFNγR, Dectin-1 and FcγR, or a combination thereof. In certain embodiments, the intracellular signaling domain of the CAR described herein comprises the cytoplasmic domain of Dectin-1 and the cytoplasmic domain of FcγR.

In certain embodiments, the cytoplasmic domain of Dectin-1 and the cytoplasmic domain of FcγR are connected in tandem. In certain embodiments, the polynucleotide encoding the cytoplasmic domain of Dectin-1 is upstream the polynucleotide encoding the cytoplasmic domain of FcγR. In certain embodiments, the polynucleotide encoding the cytoplasmic domain of Dectin-1 is downstream the polynucleotide encoding the cytoplasmic domain of FcγR.

The cytoplasmic domain of Dectin-1 may comprise an amino acid sequence set forth in SEQ ID NO: 1, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof. In certain embodiments, the cytoplasmic domain of Dectin-1 comprise an amino acid sequence set forth in SEQ ID NO: 58, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof. In certain embodiments, the cytoplasmic domain of Dectin-1 comprise an amino acid sequence set forth in SEQ ID NO: 58.

The cytoplasmic domain of FcγR may comprise an amino acid sequence set forth in SEQ ID NO: 2, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof. In certain embodiments, the cytoplasmic domain of FcγR may comprise an amino acid sequence set forth in SEQ ID NO: 59 and/or SEQ ID NO: 60, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof. In certain embodiments, the cytoplasmic domain of FcγR may comprise an amino acid sequence set forth in SEQ ID NO: 59 and/or SEQ ID NO: 60.

In certain embodiments, the intracellular signaling domain of the CAR described herein comprises an amino acid sequence set forth in SEQ ID NO: 3, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof.

In certain embodiments, the intracellular signaling domain of the CAR described herein comprises an amino acid sequence encoded by a nucleic acid sequence set forth in SEQ ID NO: 4, or a sequence having at least 75%, 80%, 85%, 90%, 95%, or 99% identity thereto while retaining substantial biological activity thereof.

(4) Co-Stimulatory Signaling Domain

In some embodiments, the intracellular signaling domain further comprises a co-stimulatory signaling domain.

In some embodiments, the co-stimulatory signaling domain is derived from an intracellular domain of a co-stimulatory molecule.

Examples of co-stimulatory molecules include B7-H3, BAFFR, BLAME (SLAMF8), CD2, CD4, CD8 alpha, CD8 beta, CD7, CD11a, CD11b, CD11c, CD11d, CD 18, CD 19, CD27, CD28, CD29, CD30, CD40, CD49a, CD49D, CD49f, CD69, CD83, CD84, CD96 (Tactile), CD100 (SEMA4D), CD103, CD 127, CD137(4-1BB), CD150 (SLAM, SLAMF1, IPO-3), CD160 (BY55), CD162 (SELPLG), CD226 (DNAM1), CD229 (Ly9), CD244 (SLAMF4, 2B4), CEACAM1, CRTAM, CDS, OX40, PD-1, ICOS, GADS, GITR, HVEM (LIGHTR), IA4, ICAM-1, IL2R beta, IL2R gamma, IL7R alpha, ITGA4, ITGA4, ITGA6, ITGAD, ITGAE, ITGAL, ITGAM, ITGAX, ITGB1, ITGB2, ITGB7, LAT, LFA-1, LIGHT, LTBR, NKG2C, NKG2D, NKp44, NKp30, NKp46, NKp80 (KLRF1), PAG/Cbp, PSGL1, SLAMF6 (NTB-A, Ly108), SLAMF7, SLP-76, TNFR2, TRANCE/RANKL, VLA1, VLA-6, any derivative, variant, or fragment thereof, any synthetic sequence of a co-stimulatory molecule that has the same functional capability, and any combination thereof.

In some embodiment, the co-stimulatory signaling domain of the CAR described herein comprises an intracellular domain of co-stimulatory molecule CD137 (4-1BB), CD28, OX40 or ICOS.

Other Regions

In some embodiments, the CAR further comprises a signal peptide. In some embodiments, the signal peptide comprises a signal peptide of CD8 alpha. In some embodiments, the signal peptide of CD8 alpha comprises the sequence of SEQ ID NO: 5, or a sequence having at least 75%, 80%, 85%, 90%, 95%, or 99% identity thereto while retaining substantial biological activity thereof, or a sequence having 1, 2, 3, 4, 5, 6, 7, 8, 9 or 10 conservative substitutions thereof, or any functional forms thereof.

Human solid tumors develop complex and heterogenous TIME to evade immunotherapy. The existing immunotherapies (such as CAR-T cell therapy) are not effective for solid tumors. Tumor infiltrating immune suppressive DCs contribute significantly to TIME. The DC-activating CARs as described above can disrupt TIME, covert TIME into an inflammatory state, enhance the cytotoxicity and survival of engineered immune cells (e.g., CAR-T cells), and significantly promote the efficacy of the engineered immune cells (e.g., CAR-T cells) to eliminate solid tumors with TIME.

### Vector

In another aspect, the present disclosure provides a vector comprising the polynucleotide encoding the CAR as described herein. The polynucleotides encoding a CAR can be inserted into different types of vectors known in the art, for example, a plasmid, a phagemid, a phage derivative, a viral vector derived from animal virus, a cosmid, transposon, a site directed insertion vector (e.g., CRISPR, Zinc finger nucleases, TALEN), an in vitro transcribed RNA, or a suicide expression vector. In some embodiments, the vector is a DNA or RNA.

In some embodiments, the vector is an expression DNA vector (e.g., plasmid, virus). When the expression DNA vector introduced into the cell transiently, mRNA of the CAR will be transcribed in host cell. As the DNA vector and the mRNA would dilute out with cell division, the expression of the CAR would not be permanent. In one embodiment, the DNA vector can be introduced to a cell as a form of transient expression of the CAR.

In some embodiments, the vector is a viral vector. Viral vectors may be derived from, for example, retroviruses, adenoviruses, adeno-associated viruses (AAV), herpes viruses, and lentiviruses. Useful viral vectors generally contain an origin of replication functional in at least one organism, a promoter, restriction endonuclease sites, and one or more selectable markers. In some embodiments, the vector is a lentiviral vector. Lentiviral vector is particular useful for long-term, stable integration of the polynucleotide encoding the CAR into the genome of non-proliferating cells that result in stable expression of the CAR in the host cell, e.g., host T cell. In some embodiments, the vector is a lenti-Cas9 vector from Addgene.

In some embodiments, the vector is RNA (e.g., mRNA). As the RNA would dilute out with cell division, the expression of the RNA would not be permanent. In one embodiment, the in vitro transcribed RNA CAR can be introduced to a cell as a form of transient expression.

In some embodiments, the vector is a transposon-based expression vector. A transposon is a DNA sequence that can change its position within a genome. In a transposon system, the polynucleotide encoding the CAR is flanked by terminal repeat sequences recognizable by a transposase which mediates the movement of the transposon. A transposase can be co-delivered as a protein, encoded on the same vector as the CAR, or encoded on a separate vector. Non-limiting examples of transposon systems include Sleeping Beauty, Piggyback, Frog Prince, and Prince Charming.

In some embodiment, the polynucleotide is operably linked to at least one regulatory polynucleotide element in the vector for expression of the CAR. Typical vectors contain various regulatory polynucleotide elements, for example, elements (e.g., transcription and translation terminators, initiation sequences, and promoters) regulating the expression of the inserted polynucleotides, elements (e.g., origin of replication) regulating the replication of the vector in a host cell, and elements (e.g., terminal repeat sequence of a transposon) regulating the integration of the vector into a host genome. The expression of the CAR can be achieved by operably linking the polynucleotides encoding a CAR to a promoter and incorporating the construct into a vector. Both constitutive promoters (such as a CMV promoter, a SV40 promoter, and a MMTV promoter), or inducible promoters (such as a metallothionine promoter, a glucocorticoid promoter, and a progesterone promoter) are contemplated for the disclosure. In some embodiment, the vector is an expression vector, an expression vector comprises sufficient cis-acting elements for expression; other elements for expression can be supplied by the host cell or in an in vitro expression system.

In order to assess the expression of a CAR, the vector can also comprise a selectable marker gene or a reporter gene or both for identification and selection of the cells to which the vector is introduced. Useful selectable markers include, for example, antibiotic-resistance genes, such as neo and the like. Useful reporters include, for example, luciferase, beta-galactosidase, chloramphenicol acetyl transferase, secreted alkaline phosphatase, or the green fluorescent protein gene.

Chemical structures with the ability to promote stability and/or translation efficiency may also be used in an RNA. A method for generating RNA for use in transfection can involve in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the nucleic acid to be expressed, and a polyA tail typically 50-2000 bases in length. RNA so produced can efficiently transfect different kinds of cells.

RNA can be introduced into target cells using any of a number of different methods, for instance, available methods which include, but are not limited to, electroporation or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns”.

A vector can be introduced into a host cell, e.g., mammalian cell by any method known in the art, for example, by physical, chemical or biological means. Physical methods for introducing a polynucleotide into a host cell include calcium phosphate precipitation, lipofection, particle bombardment, microinjection, electroporation, and the like. Biological methods include the use of viral vectors, and especially retroviral vectors, for inserting genes into mammalian, e.g., human cells. Chemical means include colloidal dispersion systems, such as macromolecule complexes, nanocapsules, microspheres, beads, and lipid-based systems including oil-in-water emulsions, micelles, mixed micelles, and liposomes.

### Cells

In one aspect, the disclosure provides an engineered cell comprising or expressing the CAR as described here. In some embodiments, the engineered cell comprises the polynucleotide encoding the CAR, or the vector comprising the CAR polynucleotide. The engineered cell provided herein may comprise or express one or more (for example, 1, 2, 3, or more) CARs. The one or more CARs may be the same or different. In certain embodiments, the engineered cell is a dendritic cell or a precursor or progenitor cell thereof. The term “dendritic cell or a precursor or progenitor cell thereof”, as used herein, refers to a native or modified dendritic cell or a precursor or progenitor cell thereof.

Sources of Cells

The engineered cells (e.g., CAR-DCs) provided herein may be obtained from any source. In certain embodiments, the engineered cells (e.g., CAR-DCs) provided herein is derived from immune cells isolated from subjects, e.g., human subjects. In some embodiments, the immune cells are obtained from a subject of interest, such as a subject suspected of having a particular disease or condition, a subject suspected of having a predisposition to a particular disease or condition, a subject who will undergo, is undergoing, or have undergone treatment for a particular disease or condition, a subject who is a healthy volunteer or healthy donor, or from blood bank. In some embodiments, the immune cells are obtained from a cancer subject who has poor responsiveness to an immunotherapy, such as CAR-T therapy.

The cells can be autologous or allogeneic to the subject of interest. Allogeneic donor cells may not be human-leukocyte-antigen (HLA)-compatible, and thus allogeneic cells can be treated to reduce immunogenicity.

Immune cells can be collected from any location in which they reside in the subject including, but not limited to, blood, cord blood, spleen, thymus, lymph nodes, pleural effusion, spleen tissue, tumor and bone marrow. The isolated immune cells may be used directly, or they can be stored for a period of time, such as by freezing.

In some embodiments, the engineered cells are obtained by engineering a dendritic cell or a precursor or progenitor cell thereof. A dendritic cell or a precursor or progenitor cell thereof can be obtained from blood collected from a subject using any number of techniques known to the skilled artisan, such as apheresis. In some embodiments, the dendritic cell or a precursor or progenitor cell thereof is derived from peripheral blood cells (e.g., peripheral blood mononuclear cells, such as a monocyte), a bone marrow cell, an embryonic stem cell, or an induced pluripotent stem cell (iPSC).

The presence of a dendritic cell can be checked using the previously described method. For example, a dendritic cell may be identified by measuring expression of CD11c, CD80, CD86, MHC/HLA molecules, and/or CCR7 molecules, which can be detected using techniques, such as immune chemistry, immunophenotyping, flow cytometry, Elispots assays, classical tetramer staining, and intracellular cytokine staining.

### Method of Producing CAR-DCs

In another aspect, the disclosure provides a method of making an engineered cell expressing the CAR as described herein. Numerous means of generating CAR-T cells known in the art can also be applied to CAR-DC. Methods for generating CAR-T cells have been described in, for example, Zhang et al., Engineering CAR-T cells, Biomarker Research (2017) 5:22. In some embodiments, the method comprises introducing to a starting cell the vector comprising the polynucleotide encoding the CARs provided herein under conditions suitable for expression of the polynucleotide. The method provided herein may comprise one of more steps selected from: obtaining a starting cell (i.e., a cell from a source), culturing (including expanding, optionally including maturating) the starting cell, and genetically modifying the cells. The starting cell can be a dendritic cell or a precursor or a progenitor cell thereof as described above.

Genetically modifying a DC or a precursor or progenitor cell thereof can be accomplished by transducing a population of substantially homogeneous DCs with a polynucleotide encoding a CAR provided herein. In certain embodiments, a retroviral vector (e.g., a lentiviral vector) is employed for the introduction of the polynucleotide provided herein into the DCs. For example, the polynucleotide provided herein can be cloned into a lentiviral vector and expression can be driven from its endogenous promoter, from the lentiviral long terminal repeat, or from a promoter specific for a target cell type of interest. Common delivery methods for delivering viral vectors include but is not limited to, electroporation, microinjection, gene gun, and magnetofection. Placement of a presently disclosed CAR can be made at any endogenous gene locus.

Non-viral approaches can also be employed for genetic modification of a DC or a precursor or progenitor cell thereof. For example, a nucleic acid molecule can be introduced into a DC or a precursor or progenitor cell thereof by administering the nucleic acid in the presence of lipofection (Ono et al., Neuroscience Letters 17:259, 1990; Feigner et al., Proc. Natl. Acad. Sci. U.S.A. 84:7413, 1987; Staubinger et al., Methods in Enzymology 101:512, 1983; Brigham et al., Am. J. Med. Sci. 298:278, 1989), sialoorosomucoidpolylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR).

In certain embodiment, the engineered cell provided herein are prepared by transfecting polynucleotide encoding the CARs provided herein into a DC prior to administration. In certain embodiments, the engineered cell provided herein can be made by transfecting a precursor or progenitor cell of DC with the polynucleotide encoding the CARs provided herein via, for example, a viral vector, followed by differentiating the transfected cell into a DC. The engineered cells provided herein exhibit improved expression of CARs on the cell surface. The precursor or progenitor cell of a DC can be derived from peripheral blood cells (e.g., peripheral blood mononuclear cells, such as a monocyte, e.g., THP-1 cell, peripheral monocytes), a bone marrow cell. The precursor or progenitor cell of a DC can also be an embryonic stem cell, or an induced pluripotent stem cell (iPSC).

In another aspect, the present disclosure also provides a population of cells produced ex vivo by the method as described above. In certain embodiments, at least 70%, at least 75%, at least 80%, at least 85%, at least 90% or at least 95% of the population of cells express a detectable level of the CAR polypeptide provided herein. In certain embodiments, at least 85% of the population of cells express a detectable level of the CAR polypeptide provided herein.

### Method of Selecting DC-Activating CAR

In another aspect, the present disclosure also provides a method of selecting a CAR capable of activating DC. The method provided herein comprises providing a non-human animal comprising an immune suppressive tumor microenvironment. In certain embodiments, the immune suppressive tumor microenvironment is clinically relevant. As used herein, The term “clinically relevant” with respective to the immune suppressive tumor microenvironment or TIME refers to an immune suppressive tumor microenvironment characterized in one or more of the following features: 1) hypoxic and acidic, 2) enriched with negative immune regulatory cells, such as regulatory T cells, immune suppressive DC cells, tumor associated macrophage and tumor associated fibroblasts, 3) with the overexpression of immune suppressive molecules, such as PD-1, TIM3, TIGIT, LAG3, A2AR, BTLA (CD272), CTLA-4 (CD152), IDO1, IDO2, TDO, NOX2, VISTA, SIGLEC7 (CD328), PVR(CD155) and SIGLEC9 (CD329), PD-L1, PD-L2, B7-H3 (CD276), B7-H4 (VTCN1), PVR(CD155), HLA class I, sialoglycoprotein, CD112, CD113, Galectin9, CD24, and CD47; and 4) capable of suppressing the activities of the tumor-infiltrating immune cells (e.g., immune effector cells).

In certain embodiments, the non-human animal (e.g., mouse) model comprises human fetal thymus and autologous human hematopoietic stem cells (e.g., autologous human CD34+ hematopoietic stem cells, for example, autologous human fetal liver CD34+ hematopoietic stem cells). The term “autologous” as used herein may refer to that the human hematopoietic stem cells and the human fetal thymus are generated from the same fetal source. In certain embodiments, the non-human animal (e.g., mouse) model is injected with about 1×10⁵ to about 5×10⁵ autologous human hematopoietic stem cells (e.g., autologous human CD34+ hematopoietic stem cells, for example, autologous human fetal liver CD34+ hematopoietic stem cells). In certain embodiments, the non-human animal (e.g., mouse) model comprises a sustained human immune system comprising human lymphohematopoietic cells, such as T cells (e.g., CD3⁺ T cells), B cells (e.g., CD19⁺ B cells) and optionally dendritic cells (DCs), which allows normal human T cell maturation in the presence of autologous human leukocyte antigens (HLAs) inside a human thymus environment. In certain embodiments, the non-human animal is a rodent, such as a rat or a mouse.

In certain embodiments, the non-human animal comprises an immune suppressive microenvironment, for example, an immune suppressive tumor microenvironment. In certain embodiments, the immune suppressive tumor microenvironment comprises a tumor and/or tumor infiltrating immune cells expressing an immune inhibitory molecule. The immune inhibitory molecule can be selected from the group consisting of PD-1, TIM3, TIGIT, LAG3, A2AR, BTLA (CD272), CTLA-4 (CD152), IDO1, IDO2, TDO, NOX2, VISTA, SIGLEC7 (CD328), PVR(CD155) and SIGLEC9 (CD329), PD-L1, PD-L2, B7-H3 (CD276), B7-H4 (VTCN1), PVR(CD155), HLA class I, sialoglycoprotein, CD112, CD113, Galectin9, CD24, and CD47. In certain embodiments, the immune inhibitory molecule is CTLA-4 and/or PD-L1. In certain embodiments, the tumor comprises a cell expressing CTLA4-Ig and/or PD-L1.

The method provided herein further comprises: administering a dendritic cell expressing a candidate CAR to the non-human animal described above, detecting a marker for the dendritic cell activation that comprises, for example, improved infiltration to the immune suppressive tumor microenvironment, improved survival rate, and/or enhanced function in inducing activation of immune cells (e.g., T cell, a Natural Killer (NK) cell, a NKT cell, a B cell, a macrophage cell, an eosinophil or a neutrophil) when compared to a reference DC, and selecting the candidate CAR as a CAR capable of activating DCs. In certain embodiments, the immune cell is a T cell selected from the group consisting of CD4+ T cell, CD8+ T cell, cytotoxic T cell, terminal effector T cell, memory T cell, naïve T cell, natural killer T cell, gamma-delta T cell, cytokine-induced killer (CTK) T cell, and tumor infiltrating lymphocyte. In certain embodiments, the immune cell is autologous or allogeneic. In certain embodiments, the immune cell is a modified immune cell (e.g., CAR-T cells) or a native immune cell. In certain embodiments, the modified immune cell (e.g., CAR-T cells) is administered in combination with the dendritic cell expressing the candidate CAR.

The method of selecting DC-activating CARs involving a non-human animal with clinically relevant TIME provides more clinically relevant DC-activating CARs or CAR-DCs. In other words, the DC-activating CARs or CAR-DCs selected by the method provided herein can not only activate DCs in an animal model but can also be expected to activate DCs under clinical settings, which can hardly be achieved so far by conventional animal models due to the increased complexity and heterogeneity of the tumor microenvironment in human patients as compared to the conventional animal models.

### Pharmaceutical Composition

In another aspect, the present disclosure also provides a pharmaceutical composition comprising the polynucleotide encoding the CARs provided herein and a pharmaceutically acceptable medium. In another aspect, the present disclosure also provides a pharmaceutical composition comprising the CAR polypeptide provided herein and a pharmaceutically acceptable medium. In another aspect, the present disclosure also provides a pharmaceutical composition comprising the vector delivering the polynucleotide encoding the CARs provided herein and a pharmaceutically acceptable medium. In another aspect, the present disclosure also provides a pharmaceutical composition comprising the population of the engineered cells (e.g., CAR-DCs) provided herein and a pharmaceutically acceptable medium. As used herein, the term “pharmaceutical composition” refers to a composition formulated for pharmaceutical use.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

A “pharmaceutically acceptable medium” refers to an ingredient in a pharmaceutical formulation, other than an active ingredient, which is biologically acceptable and nontoxic to a subject. Pharmaceutical acceptable medium for use in the pharmaceutical compositions disclosed herein may include, for example, pharmaceutically acceptable liquid, gel, or solid carriers, aqueous or nonaqueous vehicles, antimicrobial agents, buffers, antioxidants, isotonic agents, suspending/dispending agents, sequestering or chelating agents, diluents, adjuvants, excipients, or non-toxic auxiliary substances, or various combinations thereof.

The pharmaceutical compositions of the present disclosure can be prepared using various techniques known in the art, see, for example, Remington, The Science And Practice of Pharmacy (21st ed. 2005). Briefly, the engineered cells or a population thereof is admixed with a suitable medium prior to use or storage. Suitable pharmaceutically acceptable medium generally comprise inert substances that help in: 1) administering the pharmaceutical composition to a subject, 2) processing the pharmaceutical compositions into deliverable preparations, and/or 3) storing the pharmaceutical composition prior to administration. In certain embodiments, the pharmaceutically acceptable medium comprises agents that can stabilize, optimize or alter the form, consistency, viscosity, pH, pharmacokinetics, and/or solubility of the formulation. Such agents include, without limitation, buffering agents, wetting agents, emulsifying agents, diluents, encapsulating agents, and skin penetration enhancers, for example, saline, buffered saline, dextrose, arginine, sucrose, water, glycerol, ethanol, sorbitol, dextran, sodium carboxymethyl cellulose, and combinations thereof.

Exemplary pharmaceutically acceptable medium include sugars (e.g., lactose, glucose and sucrose), starches (e.g., corn starch and potato starch), cellulose and derivatives thereof (e.g., sodium carboxymethyl cellulose, methylcellulose, ethyl cellulose, microcrystalline cellulose and cellulose acetate), powdered tragacanth, malt, gelatin, lubricating agents (e.g., magnesium stearate, sodium lauryl sulfate and talc), excipients (e.g., cocoa butter and suppository waxes), oils (e.g., peanut oil, cottonseed oil, safflower oil, sesame oil, olive oil, corn oil and soybean oil, glycols (e.g., propylene glycol), polyols (e.g., glycerin, sorbitol, mannitol and polyethylene glycol (PEG)), esters (e.g., ethyl oleate and ethyl laurate), agar, buffering agents (e.g., magnesiums hydroxide and aluminum hydroxide), alginic acid, pyrogen-free water, isotonic saline, Ringer's solution, ethyl alcohol, pH buffered solutions, polyesters, polycarbonates, polyanhydrides, bulking agents (e.g., polypeptides and amino acids, serum alcohols (e.g., ethanol), (sterile) phosphate-buffered saline, Ringer's solution, dextrose solution and other non-toxic compatible substances used in pharmaceutical formulations.

The pharmaceutical compositions provided herein can be administered systemically or directly to a subject for inducing and/or enhancing an immune response to an antigen and/or treating and/or preventing a neoplasm, pathogen infection, or infectious disease. In certain embodiments, the pharmaceutical compositions provided herein are directly injected into a tumor or organ of interest. In other embodiments, the pharmaceutical compositions provided herein are administered indirectly to the organ of interest, for example, by administration into the circulatory system (e.g., the tumor vasculature).

The pharmaceutical compositions provided herein may comprise at least a population of about 1×10⁵, about 2×10⁵, about 3×10⁵, about 4×10⁵ or about 5×10⁵ engineered cells (e.g., CAR-DCs). Those skilled in the art can readily determine the percentage of the engineered cells (e.g., CAR-DCs) provided herein in a population using various well-known methods, for example, fluorescence activated cell sorting (FACS). Suitable ranges of the percentage of the engineered cells (e.g., CAR-DCs) provided herein in a population (also referred as “purity”) may be about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, or about 95% to about 100%.

In certain embodiment, the recipient is administered at least 1×10³ cells/kg of bodyweight, at least 5×10³ cells/kg of bodyweight, at least 1×10⁴ cells/kg of bodyweight, at least 5×10⁴ cells/kg of bodyweight, at least 1×10⁵ cells/kg of bodyweight, at least 5×10⁵ cells/kg of bodyweight, at least 1×10⁶ cells/kg of bodyweight, at least 5×10⁶ cells/kg of bodyweight, at least 1×10⁷ cells/kg of bodyweight, at least 5×10⁷ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 6×10⁸ cells/kg of bodyweight. A person skilled in the art would understand that dosage of the pharmaceutical compositions provided herein may be determined based on various factors of the recipient, such as size, age, sex, weight, and condition. Dosages can be readily determined by a person skilled in the art from this disclosure and the knowledge in the art. The person skilled in the art can readily determine the number of the engineered cells provided herein and the amount of optional additives, vehicles, medium and/or carriers in compositions and to be administered in methods of the present disclosure. Typically, additives, if any, are present in an amount of 0.001 to 50% (weight) solution in phosphate buffered saline, and the active ingredient (e.g., the modified/recombinant cells provided herein) is present in the order of micrograms to milligrams, such as about 0.0001 to about 5 wt %, preferably about 0.0001 to about 1 wt %, still more preferably about 0.0001 to about 0.05 wt % or about 0.001 to about 20 wt %, preferably about 0.01 to about 10 wt %, and still more preferably about 0.05 to about 5 wt %. It would be preferred to determine the toxicity of a certain dosage, such as by determining the lethal dose (LD) and LD50 in a suitable animal model (e.g., a mouse). It would also be preferred to determine the timing of administering the composition(s), which elicit a suitable response. Such determinations do not require undue experimentation from the knowledge of the person skilled in the art and the present disclosure.

The pharmaceutical compositions provided herein can be administered by, for example, injection (e.g., systemic injection, localized injection, intravenous injection, intralymphatic injection) or catheter. In certain embodiments, the pharmaceutical compositions provided herein can be administered subcutaneously, intradermally, intratumorally, intramedullary, or intraperitoneally. In one embodiment, the cell compositions of the present disclosure are preferably administered by intravenous injection. The administration can be autologous or heterologous. For example, the engineered cells (e.g., CAR-DCs) can be obtained by modifying the starting cells from one subject and administered to the same subject or a different subject. The pharmaceutical compositions provided herein can be formulated in a unit dosage injectable form (e.g., solution, suspension, emulsion) for administration. The administration of the pharmaceutical compositions provided herein can occur as a single event or over a time course of treatment, such as daily, weekly, bi-weekly, or monthly. The pharmaceutical compositions provided herein can be administered in combination with (e.g., before, after, or simultaneously with) another agent, such as a chemotherapeutic agent, another form of immune therapy (e.g., CAR-T therapy), or radiation therapy. Simultaneous administration can occur through the administration of separate compositions, each containing the engineered cell (e.g., CAR-DC) provided herein and another agent, such as a chemotherapeutic agent, another form of immune therapy (e.g., CAR-T therapy), or radiation therapy. Simultaneous administration can occur through the administration of one composition containing the engineered cell (e.g., CAR-DC) provided herein and another agent, such as a chemotherapeutic agent, another form of immune therapy (e.g., CAR-T therapy), or radiation therapy.

### Kit

In another aspect, the present disclosure also provides a kit comprising the engineered cells (e.g., CAR-DCs) provided herein. In another aspect, the present disclosure also provides a kit comprising the polypeptides provided herein, the polynucleotides or expression vectors provided herein for use in generating CAR-DCs provided herein.

In some embodiments, the kits of the present disclosure comprise written instructions for the use of the kit. In certain embodiments, the instructions include at least one of the following: clinical studies, precautions, warnings, and/or references. The instructions can be either printed directly on the container (when present) or provided in the container or with the container as a label applied to the container, or as a separate sheet, pamphlet, card, or folder. Suitable containers include, for example, bottles, syringes, vials, and test tubes. The containers can be formed from a variety of materials such as plastic or glass. In certain embodiments, the container holds the pharmaceutical composition provided herein and have a sterile access port.

In certain embodiments, the kit further comprises a second container comprising a pharmaceutically acceptable medium as described above. In certain embodiments, the kit further comprises other materials that are commercially desirable or user friendly, such as other diluents, buffers, needles, filters, syringes, and package inserts with instructions for use.

### Method of Uses

The present disclosure also provides various uses of the engineered cells (e.g., CAR-DCs) provided herein.

General Uses

In one aspect, the present disclosure provides a method for treating a disease or pathological condition in a patient comprising administering a therapeutically effective amount of the engineered cell provided herein to the patient. In some embodiments, the method for treating a disease or pathological condition comprises providing DCs isolated from or derived from the cells (e.g., a peripheral blood cell, a bone marrow cell, an embryonic stem cell) isolated from a subject or derived from an iPSC, engineering the DCs to express the CAR as provided herein, and transfuse the engineered cells (e.g., CAR-DCs) back into the subject. In some embodiments, the method for treating a disease or pathological condition comprises providing a precursor or progenitor cell of a DC (e.g., a peripheral blood cell, a bone marrow cell, an embryonic stem cell, or an iPSC), differentiating and engineering the precursor or progenitor cell to express the CAR as provided herein, and transfuse the differentiated and engineered cells (e.g., CAR-DCs) back into the subject. In some embodiments, the method for treating a disease or pathological condition comprises providing a precursor or progenitor cell of a DC (e.g., a peripheral blood cell, a bone marrow cell, an embryonic stem cell, or an iPSC), engineering the precursor or progenitor cell to express the CAR as provided herein, differentiating the engineered precursor or progenitor cell into a DC expressing CAR as provided herein, and transfuse the DC expressing CAR as provided herein (e.g., CAR-DCs) back into the subject.

In some embodiments, the disease is cancer.

In some embodiments, the cancer is a solid cancer selected from the group consisting of adrenal cancer, bone cancer, brain cancer, breast cancer, colorectal cancer, esophageal cancer, eye cancer, gastric cancer, head and neck cancer, kidney cancer, liver cancer, lung cancer, non-small cell lung cancer, bronchioloalveolar cell lung cancer, mesothelioma, head and neck cancer, squamous cell carcinoma, melanoma, oral cancer, ovarian cancer, cervical cancer, penile cancer, prostate cancer, pancreatic cancer, skin cancer, sarcoma, testicular cancer, thyroid cancer, uterine cancer, vaginal cancer. In some embodiments, the cancer is a hematologic malignancy selected from the group consisting of diffuse large B-cell lymphoma (DLBCL), extranodal NK/T-cell lymphoma, HHV8-associated primary effusion lymphoma, plasmablastic lymphoma, primary CNS lymphoma, primary mediastinal large B-cell lymphoma, T-cell/histiocyte-rich B-cell lymphoma, Hodgkin's lymphoma, non-Hodgkin's lymphoma, Waldenstrom's macroglobulinemia, multiple myeloma (MM).

In some embodiments, the subject having cancer is poorly responsive to a cancer therapy (e.g., immunotherapy).

The term “immunotherapy” as used herein, refers to a type of therapy that stimulates immune system to fight against disease such as cancer or that boosts immune system in a general way. Immunotherapy includes passive immunotherapy by delivering agents with established tumor-immune reactivity (such as effector cells) that can directly or indirectly mediate anti-tumor effects and does not necessarily depend on an intact host immune system (such as an antibody therapy or CAR-T cell therapy). Immunotherapy can further include active immunotherapy, in which treatment relies on the in vivo stimulation of the endogenous host immune system to react against diseased cells with the administration of immune response-modifying agents.

Examples of immunotherapy include, without limitation, checkpoint modulators, adoptive cell transfer, cytokines, oncolytic virus and therapeutic vaccines.

Checkpoint modulators can interfere with the ability of cancer cells to avoid immune system attack, and help the immune system respond more strongly to a tumor. Immune checkpoint molecule can mediate co-stimulatory signal to augment immune response or can mediate co-inhibitory signals to suppress immune response. Examples of checkpoint modulators include, without limitation, modulators of PD-1, PD-L1, PD-L2, CTLA-4, TIM-3, LAG3, A2AR, CD160, 2B4, TGF β, VISTA, BTLA, TIGIT, LAIR1, OX40, CD2, CD27, CD28, CD30, CD40, CD47, CD122, ICAM-1, IDO, NKG2C, SLAMF7, SIGLEC7, NKp80, CD160, B7-H3, LFA-1, 1COS, 4-1BB, GITR, BAFFR, HVEM, CD7, LIGHT, IL-2, IL-7, IL-15, IL-21, CD3, CD16 and CD83. In certain embodiments, the immune checkpoint modulator comprises a PD-1/PD-L1 axis inhibitor.

Adoptive cell transfer, which is a treatment that attempts to boost the natural ability of the T cells to fight cancer. In this treatment, T cells are taken from the patient, and are expanded and activated in vitro. In certain embodiments, the T cells are modified in vitro to CAR-T cells. T cells or CAR-T cells that are most active against the cancer are cultured in large batches in vitro for 2 to 8 weeks. During this period, the patients will receive treatments such as chemotherapy and radiation therapy to reduce the body's immunity. After these treatments, the in vitro cultured T cells or CAR-T cells will be given back to the patient. In certain embodiments, the immunotherapy is CAR-T therapy.

### Disruption of TIME

In one aspect, the present disclosure provides a method of disrupting TIME (for example, converting TIME into an inflammatory state) using the CAR-DCs or a population thereof provided herein.

In another aspect, the present disclosure also provides a method of inducing proliferation of immune cells, prolonging the survival of immune cells, and/or increasing expression and/or secretion of immune stimulating cytokines from immune cells in an immune suppressive microenvironment. The immune stimulating cytokines can be one or more of TNF-α, IFN-β, IFN-γ, IL-1, IL-2, IL-4, IL-6, IL-8, IL-10, IL-12, IL-18 and granulocyte-macrophage colony stimulating factor. The method comprises contacting the immune suppressive microenvironment with the engineered cell (e.g., CAR-DCs) provided herein. The immune cell can be a T cell, a Natural Killer (NK) cell, a NKT cell, a B cell, a macrophage cell, an eosinophil or a neutrophil. In certain embodiments, the immune cell is a T cell, selected from the group consisting of CD4+ T cell, CD8+ T cell, cytotoxic T cell, terminal effector T cell, memory T cell, naïve T cell, natural killer T cell, gamma-delta T cell, cytokine-induced killer (CIK) T cell, and tumor infiltrating lymphocyte. In certain embodiments, the immune cell is an unmodified immune cell. In certain embodiments, the immune cell is a modified immune cell. The unmodified or modified immune cell can be autologous or allogeneic. In certain embodiments, the modified immune cell is a CAR-T cell. In certain embodiments, the CAR-T cell is derived from the same source (e.g., peripheral blood of a subject) as the engineered cell (e.g., CAR-DC) provided herein.

In certain embodiments, the immune suppressive microenvironment is an immune suppressive tumor microenvironment. The immune suppressive tumor microenvironment has been described in the section titled “Dendritic Cell (DC)-Activating Chimeric Antigen Receptor (CAR)”. In certain embodiments, the immune suppressive tumor microenvironment comprises a tumor and/or a tumor infiltrating immune cell expressing an immune inhibitory molecule, for example, selected from the group consisting of PD-1, TIM3, TIGIT, LAG3, A2AR, BTLA (CD272), CTLA-4 (CD152), IDO1, IDO2, TDO, NOX2, VISTA, SIGLEC7 (CD328), PVR(CD155) and SIGLEC9 (CD329), PD-L1, PD-L2, B7-H3 (CD276), B7-H4 (VTCN1), PVR(CD155), sialoglycoprotein, CD112, CD113, Galectin9, CD24, and CD47. In certain embodiments, the immune inhibitory molecule is CTLA-4 and/or PD-L1. In certain embodiments, the tumor comprises a cell expressing CTLA4-Ig and/or PD-L1.

Improving Efficacy of Adoptive Cell Therapy (e.g., CAR-T Therapy)

In another aspect, the present disclosure provides a method for improving efficacy of adoptive cell therapy in treating cancer in a subject in need thereof. The method comprises administering a therapeutically effective amount of the pharmaceutical composition provided herein. In certain embodiments, the method provided herein further comprises administering a pharmaceutical composition comprising a population of modified immune cells.

The adoptive cell therapy comprises adoptive transfer of modified immune cells, such as immune cells expressing synthetic receptors (e.g., CARs or TCRs) on the cell surface. The modified immune cell can be a T cell, a Natural Killer (NK) cell, a NKT cell, a B cell, a macrophage cell, an eosinophil or a neutrophil. In certain embodiments, the immune cell is a T cell, selected from the group consisting of CD4+ T cell, CD8+ T cell, cytotoxic T cell, terminal effector T cell, memory T cell, naïve T cell, natural killer T cell, gamma-delta T cell, cytokine-induced killer (CIK) T cell, and tumor infiltrating lymphocyte. The modified immune cell can be autologous or allogeneic. In certain embodiments, the modified immune cell is a CAR-T cell. In certain embodiments, the CAR-T cell is derived from the same source (e.g., peripheral blood of a subject) as the engineered cell (e.g., CAR-DC) provided herein.

In some embodiments, the cancer is a solid tumor, or a hematologic malignancy as described above.

In some embodiments, the subject having cancer is poorly responsive to a cancer therapy (e.g., immunotherapy) as described above.

Combination Therapy

In another aspect, the present disclosure provides a combination therapy using the engineered cells (e.g., CAR-DCs) provided herein and a second agent.

In certain embodiments, the second agent is a population of modified immune cells as described above, such as CAR-T cells. In certain embodiments, the CAR-T cell is derived from the same source (e.g., peripheral blood of a subject) as the engineered cell (e.g., CAR-DC) provided herein. In certain embodiments, the ratio of engineered cells (e.g., CAR-DCs) and CAR-T cells provided in the combination therapy is in a range of about 1:1 to 1:10.

In certain embodiments, the engineered cells (e.g., CAR-DCs) provided herein and the CAR-T cells are in the same pharmaceutical composition. In certain embodiments, the engineered cells (e.g., CAR-DCs) provided herein and the CAR-T cells are in two separate pharmaceutical compositions. In certain embodiments, the engineered cells (e.g., CAR-DCs) provided herein are administered to a subject in need thereof before, simultaneously or after administration of CAR-T cells.

In certain embodiments, the second agent is an agent that inhibits immunosuppressive pathways, including but not limited to, inhibitors of TGF-β, interleukin 10 (IL-10), adenosine, VEGF, indoleamine 2,3 dioxygenase 1 (IDO1), indoleamine 2,3-dioxygenase 2 (IDO2), tryptophan 2-3-dioxygenase (TDO), lactate, hypoxia, arginase, and prostaglandin E2. The second agent can also be a T-cell checkpoint inhibitor, including but not limited to, anti-CTLA4 antibody (e.g., Ipilimumab) anti-PD1 antibody (e.g., Nivolumab, Pembrolizumab, Cemiplimab), anti-PD-L1 antibody (e.g., Atezolizumab, Avelumab, Durvalumab), anti-PD-L2 antibody, anti-BTLA antibody, anti-LAG3 antibody, anti-TIM3 antibody, anti-VISTA antibody, anti-TIGIT antibody, and anti-KIR antibody.

In certain embodiments, the second agent is a T cell agonist, including but not limited to, antibodies that stimulate CD28, ICOS, OX-40, CD27, 4-1BB, CD137, GITR, and HVEM. In certain embodiment, the second agent is a therapeutic oncolytic virus, including but not limited to, rhabdoviruses, retroviruses, paramyxoviruses, picornaviruses, reoviruses, parvoviruses, adenoviruses, herpesviruses, and poxviruses.

In certain embodiments, the second agent is an immunostimulatory agent, such as toll-like receptors agonists, including but not limited to, TLR3, TLR4, TLR7 and TLR9 agonists. In certain embodiments, the second agent is a stimulator of interferon gene (STING) agonists, such as cyclic GMP-AMP synthase (cGAS).

In certain embodiments, the CAR-DCs or a population of the CAR-DCs provided herein are administered to a subject in need thereof in conjunction with, e.g., before, simultaneously or following, any number of relevant treatment modalities, including but not limited to, treatment with cytokines, or expression of cytokines from within the CAR-DCs, that enhance dendritic cell or T-cell proliferation and persistence and, include but not limited to, Flt3L, IL-2, IL-7, and IL-15 or analogues thereof.

In some embodiments, the treatment method further comprises administering an agent that reduces of ameliorates a side effect associated with the administration of the engineered cells. Exemplary side effects include cytokine release syndrome (CRS), and hemophagocytic lymphohistiocytosis (HLH, also termed macrophage activation syndrome (MAS)). In certain embodiments, the agent administered to treat the side effects comprises an agent that neutralizes soluble factors such as IFN-gamma, IFN-alpha, IL-2 and IL-6. Exemplary agents include, without limitation, an inhibitor of TNF-alpha (e.g., entanercept) and an inhibitor of IL-6 (e.g., tocilizumab).

### EXAMPLE

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### Example 1

### Generation of CAR to Specifically Activate DCs

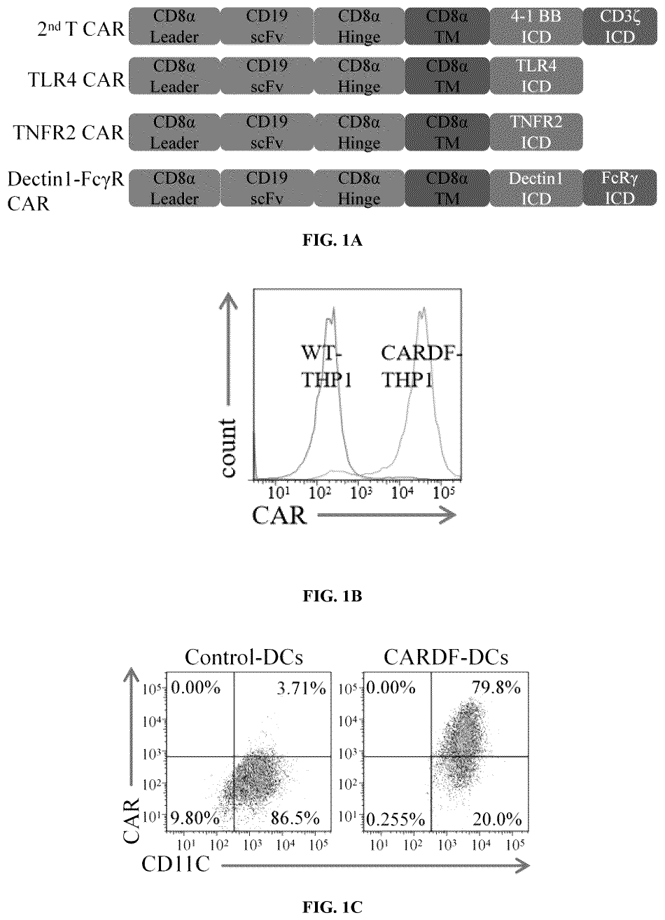

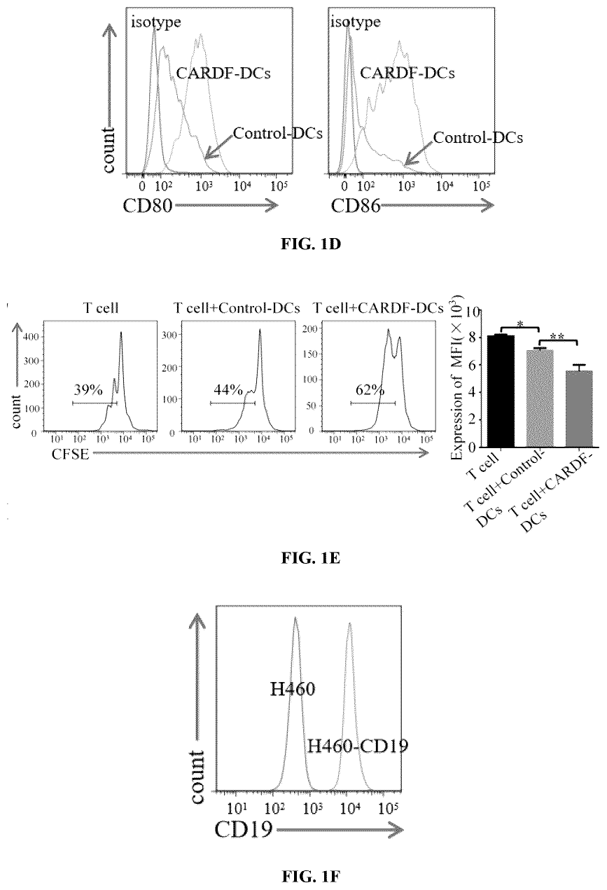

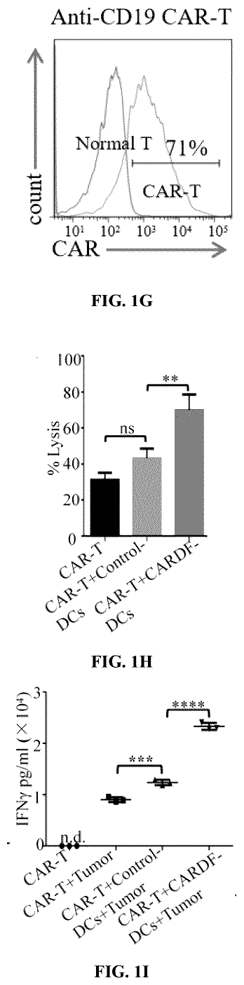

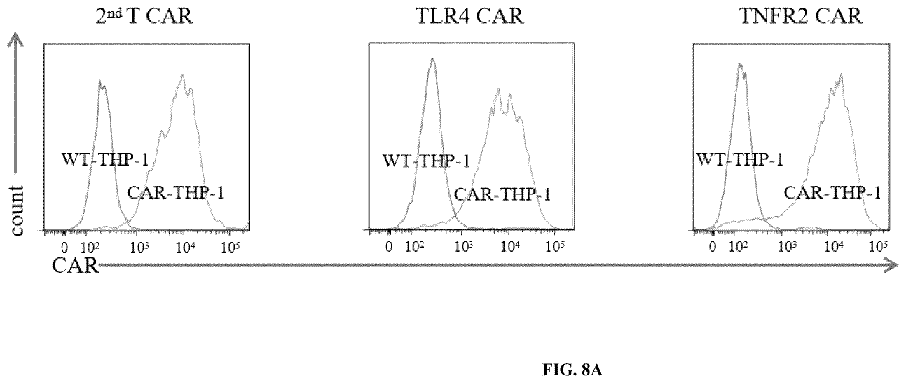

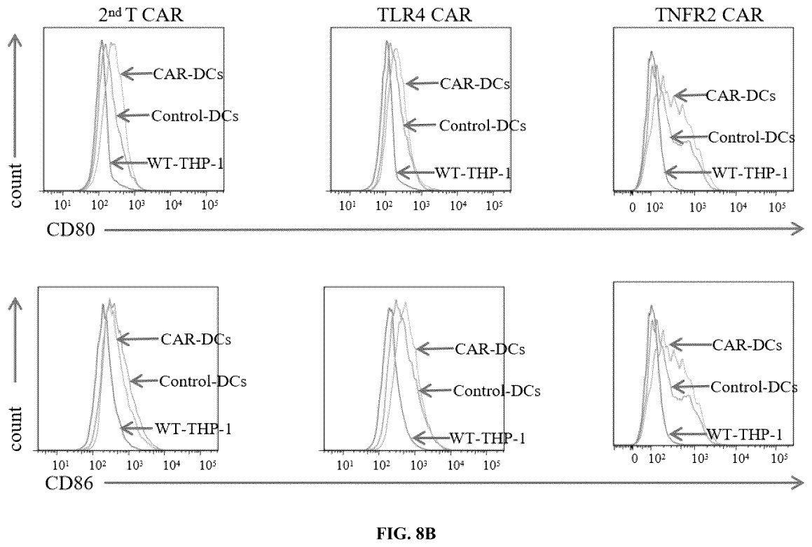

Due to distinct pathways involved in activating T cells and DCs, we reasoned that the typical CAR molecules of CAR-T cells would fail to activate DCs (FIG. 1A, FIG. 8A and FIG. 8B). Therefore, we evaluated new CARs incorporating the DC-activating pathways such as TLR4, TNFR2, Dectin-1 and FcγR. We initially tested CAR structures that consist of anti-human CD19 scFv and intracellular activating domains of TLR4, TNFR2, Dectin-1, and FcγR in DCs derived from THP-1 cells, a human monocytic leukemia cell line that can be differentiated into functional DCs with cytokine cocktails (C. Berges et al., A cell line model for the differentiation of human dendritic cells. Biochem. Biophys. Res. Commun. 333, 896-907 (2005)). CARs with TLR4 or TNFR2 tail could not effectively activate DCs, indicating that the stimulatory signals conferred by TLR4 or TNFR tail alone is not sufficient for DC activation (FIG. 8A and FIG. 8B). The expression of a CAR consisting of anti-human CD19 scFv with the tandem fusion of the cytoplasmic tails of Dectin1 and FcRγ in THP-1 cells did not affect their differentiation into DCs, denoted CARDF-DCs (FIGS. 1, B and C). When CARDF-DCs and control THP-1 derived DCs were exposed to H460-CD19 (FIG. 1F), CARDF-DCs expressed higher levels of co-stimulatory molecules (CD80 and CD86) compared to control DCs (FIG. 1D). In addition, CARDF-DCs could induce more robust proliferation of allogeneic T cells than control DCs (FIG. 1E). To investigate whether CARDF-DCs could activate the functions of CAR-T cells, the 2nd generation anti-CD19 CAR-T cells were cultured with H460-CD19 tumor cells in the presence of CARDF-DCs or control DCs (FIGS. 1, A and G). CAR-T cells conferred higher cytotoxicity to CD19+H460 tumor cells in the presence of CARDF-DCs than control DCs (FIG. 1H). In addition, CARDF-DCs induced higher expression levels of IFN-γ in CAR-T cells and release of Lactate Dehydrogenase (LDH) by tumor cells when compared to control DCs (FIGS. 1, I and J). These data indicate that CARDF can enhance the activities of DCs to activate CAR-T cells.

### Example 2

### CARDF can Activate DCs Derived from Normal Peripheral Monocytes

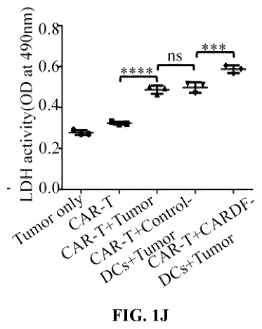

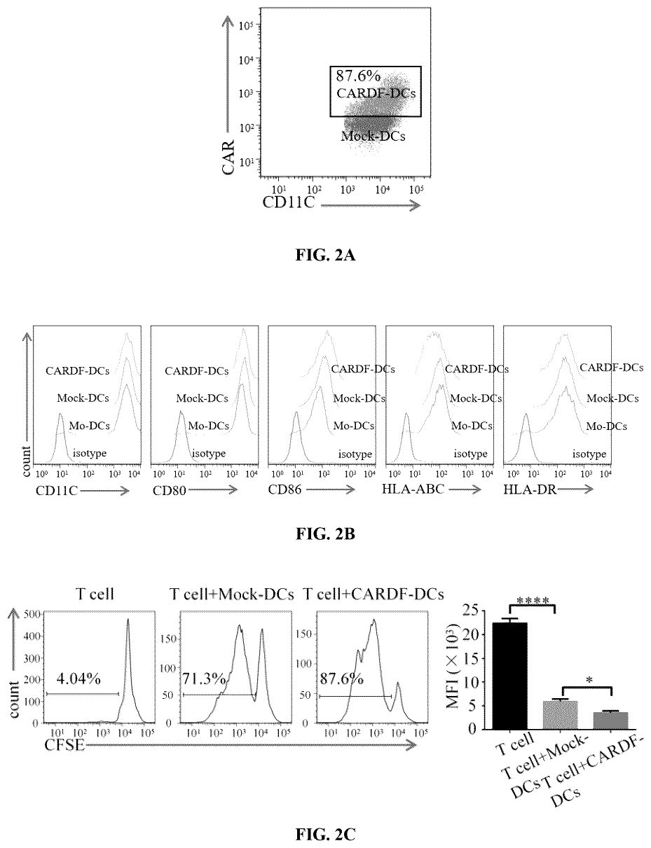

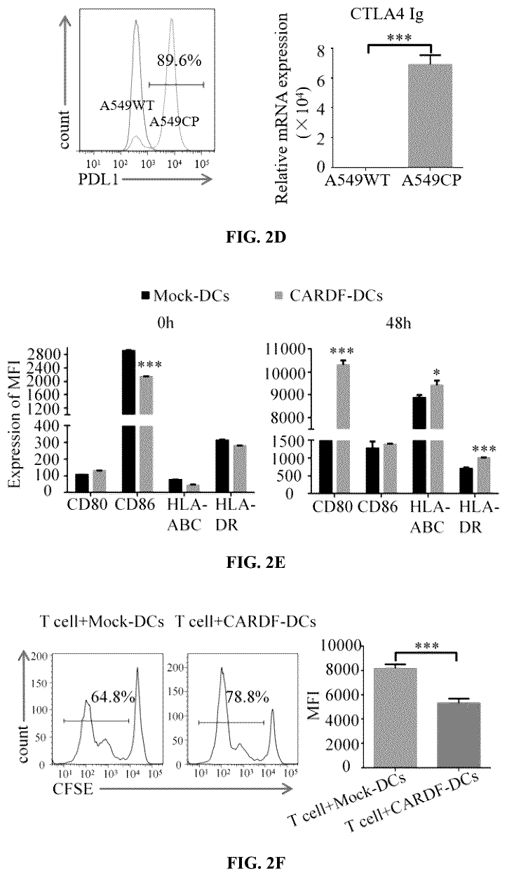

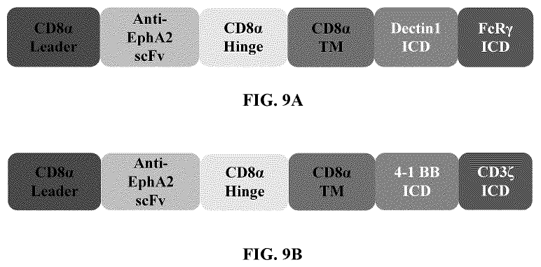

To confirm the findings of CARDF in DCs derived from THP-1 cells, we examined the impact of CARDF expression on normal DCs derived from peripheral monocytes (Mo-DCs), a common source of DCs for clinical application (J. Constantino et al., Antitumor dendritic cell-based vaccines: lessons from 20 years of clinical trials and future perspectives. Transl. Res. 168, 74-95 (2016)). Monocytes purified from PBMCs of healthy donors were transduced with the lentivirus expressing CARDF and induced to differentiate into DCs (FIG. 2A). The expression of CARDF did not affect the differentiation and maturation of Mo-DCs, with comparable surface expression levels of DC markers CD11C, CD80, CD86, HLA-ABC, and HLA-DR as control DCs (FIG. 2B). Instead of using the anti-CD19 scFv CAR that is not specific for non-engineered solid tumors, we used the antibody scFv for EphA2 (FIG. 9A), which is highly expressed by many types of solid tumors (J. Wykosky et al., The EphA2 receptor and ephrinA1 ligand in solid tumors: function and therapeutic targeting. Mol. Cancer Res. 6, 1795-1806 (2008); J. M Brannan et al., EphA2 in the early pathogenesis and progression of non-small cell lung cancer. Cancer Prev. Res. 2, 1039-1049 (2009); V. M Youngblood et al., The Ephrin-A1 EPHA2 Signaling Axis Regulates Glutamine Metabolism in HER2-Positive Breast Cancer. Cancer Res. 76, 1825-1836 (2016); M. Tandon et al., Emerging strategies for EphA2 receptor targeting for cancer therapeutics. Expert Opin Ther Targets. 15, 31-51 (2011)). To assess whether anti-EphA2 CARDF-DCs could enhance the expansion of CD3+ T cells, we cultured CFSF-labeled T cells with CARDF Mo-DCs or control Mo-DCs, which had been pre-exposed to human lung cancer A549 cells that express EphA2 for 48 hours. CARDF-DCs could induce T cell proliferation more robustly than the control Mo-DCs (FIG. 2C). In summary, our findings indicate that CARDF can activate Mo-DCs in response to the stimulation of tumor antigens.

To suppress effector T cells and promote tumor growth, previous findings have shown that the immunosuppressive TIDCs within TIME can be induced by the expression of PD-L1 and CTLA4 on the surface of solid tumor cells (J. M Tran Janco, P. Lamichhane et al., Tumor-infiltrating dendritic cells in cancer pathogenesis. J. Immunol. 194, 2985-2991 (2015); C. Fu et al., Dendritic Cells and CD8 T Cell Immunity in Tumor Microenvironment. Front Immunol. 9, 3059 (2018); C. Pfirschke et al., Tumor Microenvironment: No Effector T Cells without Dendritic Cells. Cancer cell 31, 614-615 (2017); R. A. Belderbos et al., Enhancing Dendritic Cell Therapy in Solid Tumors with Immunomodulating Conventional Treatment. Mol. Ther. Oncolytics 13, 67-81 (2019)). To evaluate the activation status of Mo-DCs in response to tumor cells expressing CTLA4-Ig and PD-L1, we constructed human lung cancer cells A549 overexpressing CTLA4-Ig and PD-L1 (A549-CP) by knock-in the expression cassette into the HPRT locus as previously described (Rong Z et al., An Effective Approach to Prevent Immune Rejection of Human ESC-Derived Allografts. Cell Stem Cell 14, 121-130 (2014)). Compared to the control A549 cells, the expression of CP was much higher in A549-CP tumor cells (FIG. 2D). When CARDF-DCs or control Mo-DCs were co-cultured with A549-CP cells for 48 hours, CARDF-DCs expressed much higher levels of CD80, HLA-ABC and HLA-DR than control Mo-DCs (FIG. 2E). When CARDF-DCs and control Mo-DCs were pre-exposed to A549-CP for 48 hours, CARDF-DCs could activate T cells more robustly than control Mo-DCs (FIG. 2F). Therefore, CARDF can activate DCs to resist tumor cell-induced immune suppression to effectively activate T cells.

### Example 3

### CARDF-DCs Activate the Cytotoxicity of CAR-T Cells In Vitro

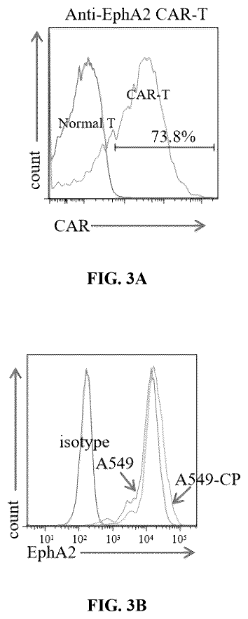

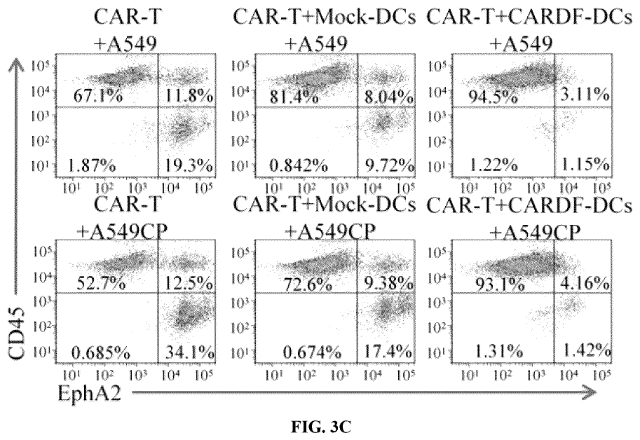

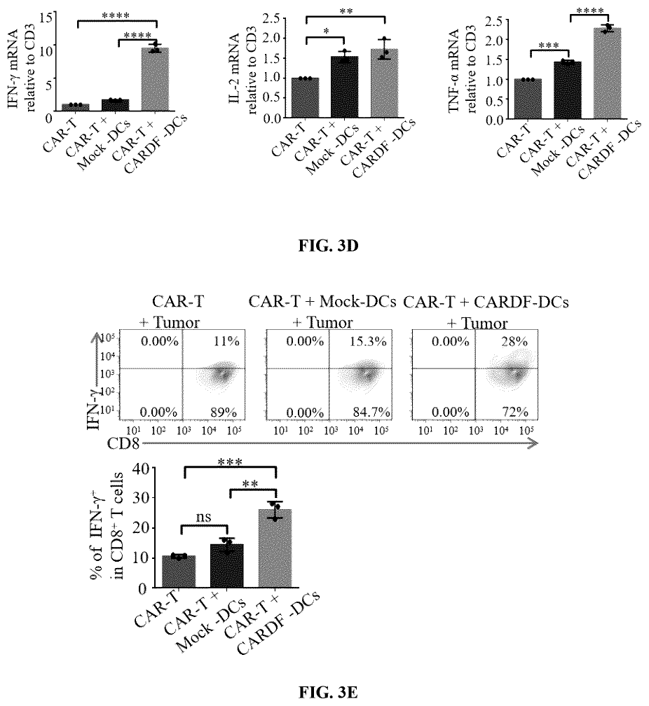

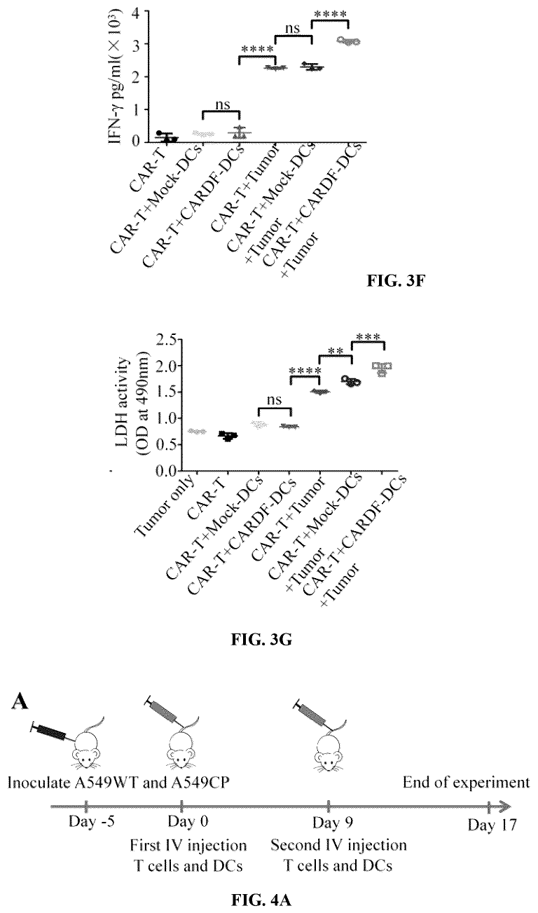

To investigate whether our CARDF-DCs increase the cytotoxicity of CAR-T cells to tumor cells, we produced anti-EphA2 CAR-T cells using the T cells from the same donor of Mo-DCs. The expression of CAR on the surface of CAR-T cells and EphA2 on the surface of A549 and A549-CP tumor cells was confirmed (FIGS. 3, A and B; FIG. 9B). To examine the cytotoxic activities of CAR-T cells activated by control Mo-DCs or CARDF-DCs, A549 and A549-CP cells were co-cultured with CAR-T cells and DCs. When compared to control Mo-DCs, CARDF-DCs significantly increased the cytotoxic activities of CAR-T cells against A549 cells (FIG. 3C). The cytolytic activity of CAR-T cells towards A549-CP cells was decreased when compared to that of A549 cells, indicating that the expression of CP suppressed the cytolytic activity of CAR-T cells. In contrast, this inhibition of cytolytic activity of CAR-T cells by the expression of CP in tumor cells was reversed by CARDF-DCs (FIG. 3C). Consistent with this finding, when compared to control Mo-DCs, the co-culture of CARDF-DCs and CAR-T cells increased the expression of IL-2, IFN-γ and TNF-α by CAR-T cells (FIG. 3D), and increased the percentage of IFN-γ+ CAR-T cells as well as the levels of IFN-γ and LDH in the supernatant (FIG. 3, E to G). Therefore, CARDF-DCs can resist the CP-mediated immune suppression to activate the cytolytic activities of CAR-T cells.

### Example 4

### CARDF-DCs are Resistant to TIME and Activate the Anti-Tumor Activities of CAR-T Cells In Vivo

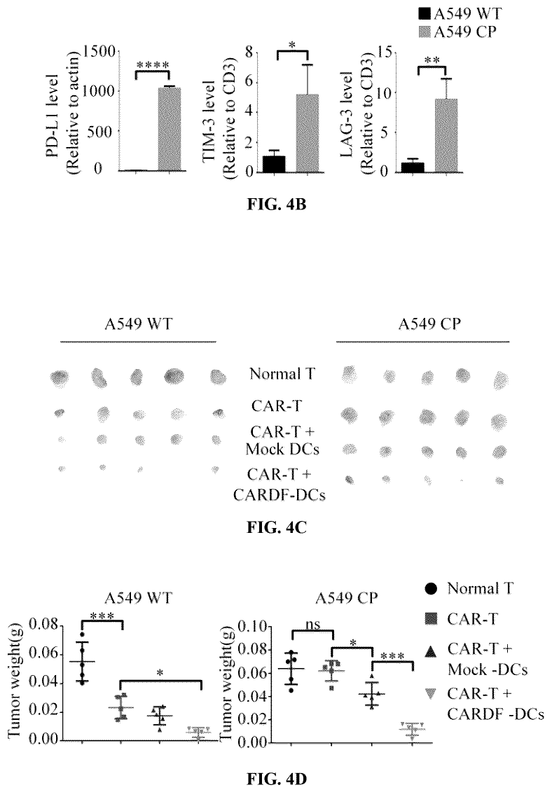

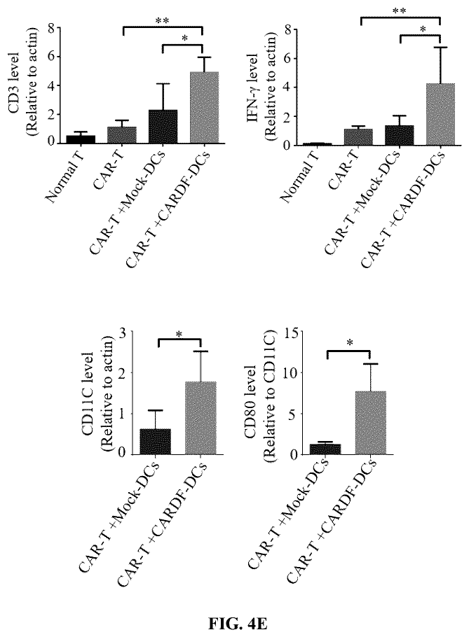

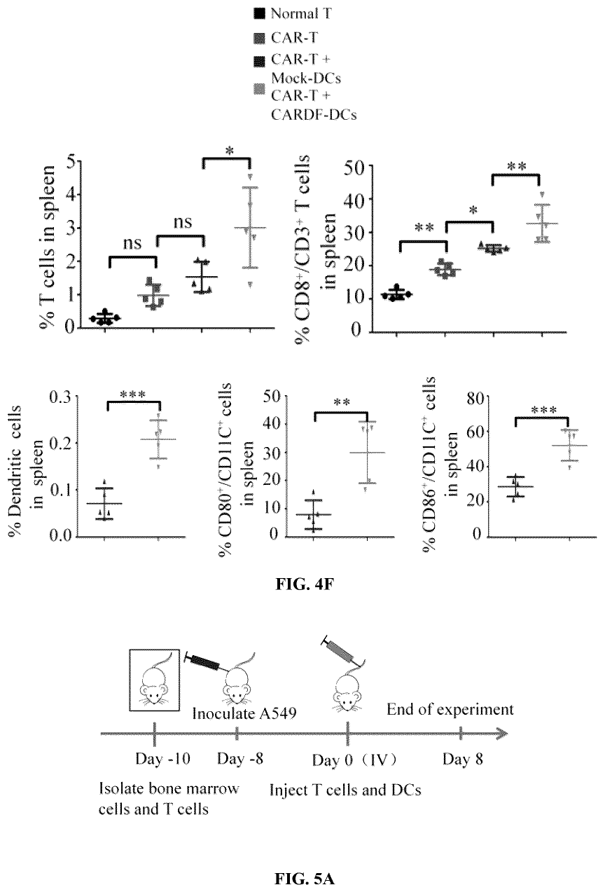

To examine the impact of CARDF-DCs on CAR-T cells in vivo, we subcutaneously injected immunodeficient NOD/SCID/IL-2γ−/− (NSG) mice with A549-WT and A549-CP tumor cells respectively. When tumors reached the palpable size, 1×107 CAR-T cells and 5×106 control Mo-DCs or CARDF-DCs were transfused intravenously respectively (FIG. 4A). In contrast to A549 tumors, A549-CP tumors developed clinically relevant TIME (FIG. 4B). Consistent with previous findings that CAR-T cells are rapidly exhausted in the solid tumors with TIME (J. L.-M. Chen et al., NR4A transcriptionfactors limit CAR T cellfunction in solid tumours. Nature 567, 530-534 (2019); J. Li et al., Chimeric antigen receptor T cell (CAR-T) immunotherapy for solid tumors: lessons learned and strategies for moving forward. J Hematol Oncol. 11, 22 (2018)), CAR-T cells effectively eliminated the A549-WT tumors but not A549-CP tumors (FIG. 4C). CAR-T cells combined with CARDF-DCs efficiently reduced A549-CP tumor burden compared to CAR-T cell treatment only (FIGS. 4, C and D). In addition, CARDF-DCs significantly prolonged the survival of T cells including CD8+ T cells in vivo and promoted the survival and activation of DCs themselves (FIGS. 4, E and F). In addition, we also detected higher expression levels of CD11C and CD80 in the tumors of the CARDF-DC treatment group (FIG. 4E), suggesting that CARDF-DCs can better infiltrate or survive longer in the CP-overexpressing tumors than the control Mo-DCs. In summary, these data suggest that CARDF-DCs can resist TIME to promote the survival and activity of CAR-T cells to suppress solid tumors.

### Example 5

### CARDF-DCs Reverse TIME to Activate CAR-T Cells to Eliminate Solid Tumor in Hu-Mice

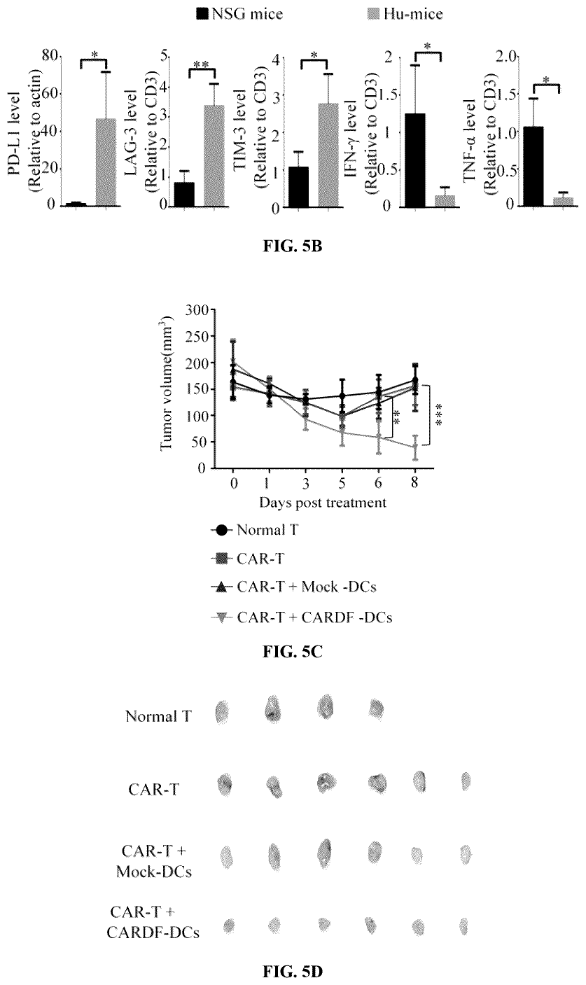

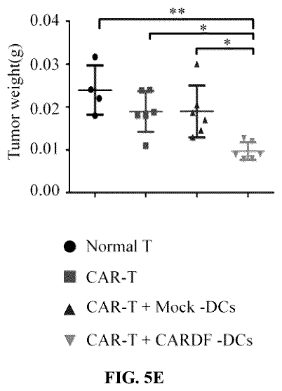

The interaction between the immune system and tumors plays key roles in the formation of TIME (M. Binnewies et al., Understanding the tumor immune microenvironment (TIME) for effective therapy. Nat Med. 24, 541-550 (2018)). Therefore, we employed the HuS model, in which human solid tumors developed clinically relevant TIME in the immune system humanized mice as previously described (Q. Li, et al., Developing Covalent Protein Drugs via Proximity-Enabled Reactive Therapeutics. Cell 182, 85-97.e16 (2020)), to further evaluate the activity of CARDF-DCs in reversing TIME of solid tumors (FIG. 5A). CARDF-DCs and CAR-T cells were derived from the bone marrow cells and T cells of Hu-mice established with the tissues of the same donor, which were also used to inoculate human lung tumors to establish HuS model. Therefore, CARDF-DCs, CAR-T cells and the immune system in HuS mice were all from the same donor. As expected, CAR-T cells with or without control Mo-DCs failed to suppress human lung tumors formed in HuS mice that develop clinically relevant TIME (FIG. 5, B to E). In contrast, CAR-T cells combined with CARDF-DCs efficiently suppressed the growth of human lung tumors formed in the same batch of Hu-mice (FIG. 5, C to E). Therefore, CARDF-DCs can resist clinically relevant TIME to activate the anti-tumor activities of CAR-T cells.

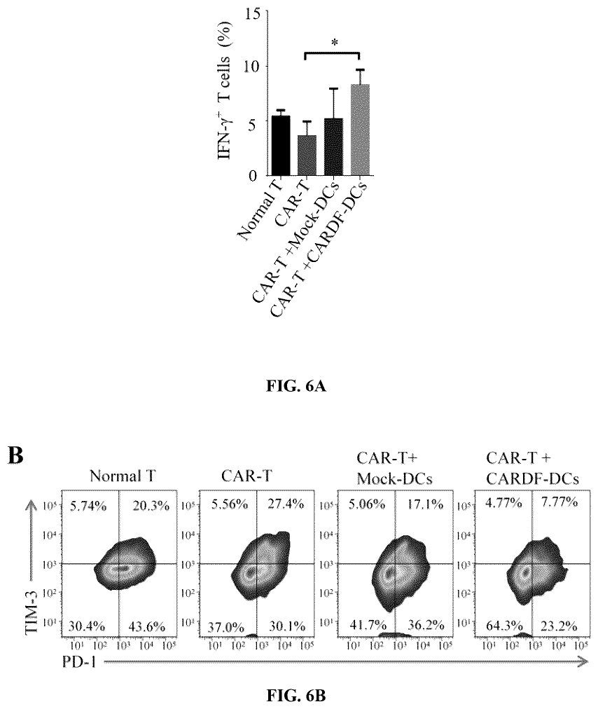

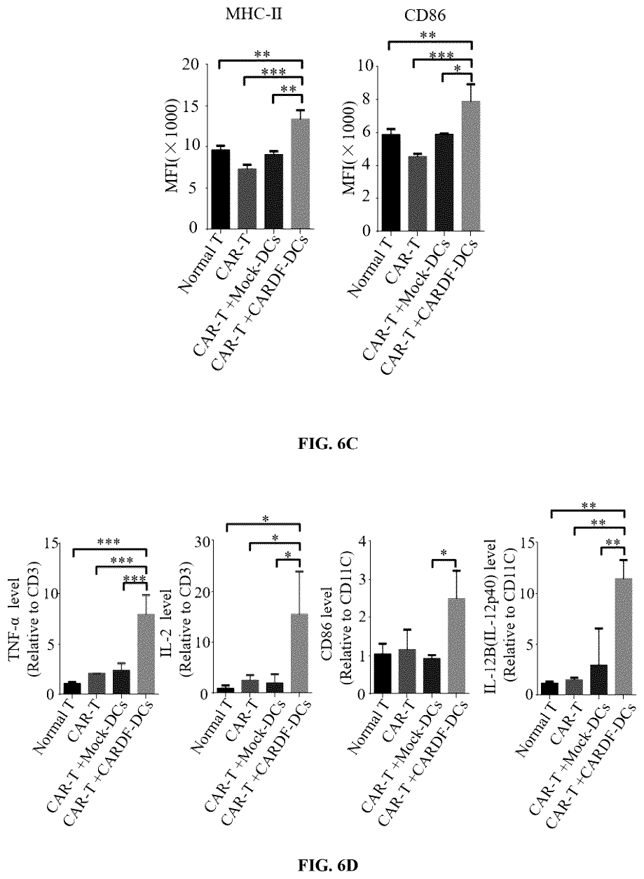

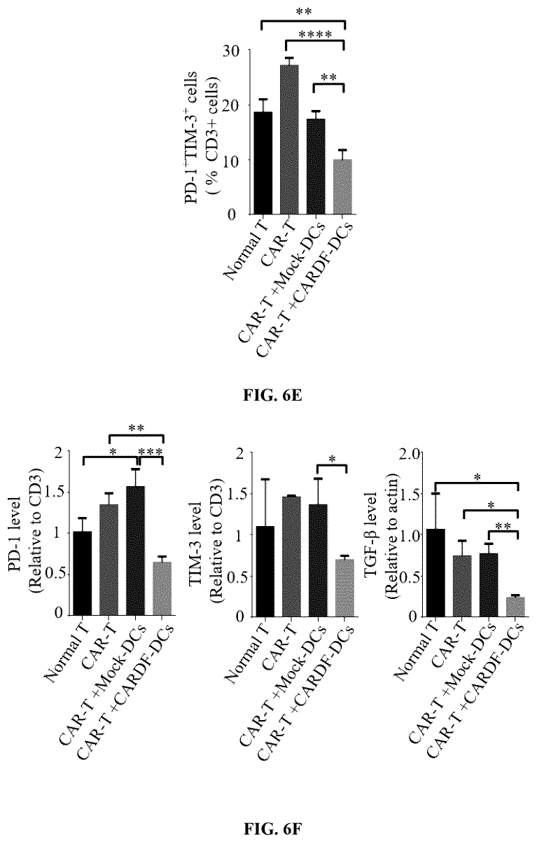

To test the hypothesis that CARDF-DCs could convert TIME of solid tumors towards a pro-inflammatory state in order to activate T cells, we examined the activation status of T cells in the periphery and in tumors. CARDF-DCs increased the percentage of IFN-γ+ T cells in the spleen (FIG. 6A) and decreased the expression of inhibitory surface receptors PD-1 and TIM-3 in splenic T cells (FIGS. 6, B and E). In addition, CARDF-DCs increased the expression of the DC activation markers CD86 and MHC-II in splenic DCs (FIG. 6C). Therefore, CARDF-DCs appeared to activate the systemic immune system. In support of the notion that CARDF-DCs could convert TIME of solid tumors towards a pro-inflammatory state, CARDF-DCs increased the intra-tumoral expression of TNF-α, IL-2, CD86, IL-12B (FIG. 6D), decreased the expression of immune checkpoint molecules PD-1, TIM-3, TGF-β (FIG. 6F) and M2 macrophage markers CD206, CD163 (FIG. 6G). These data indicate that CARDF-DCs can reverse TIME towards the pro-inflammatory conditions to activate immunity to solid tumors.

### Example 6

### CARDF-DCs have Uniform T Cell Activating Activities in Different TIME

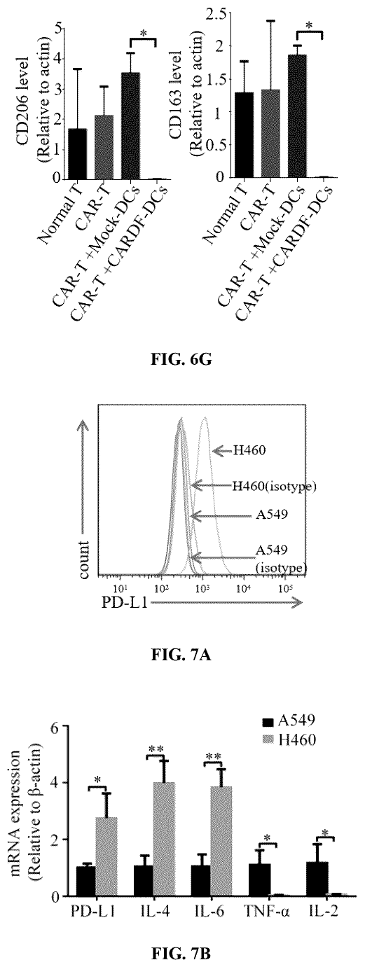

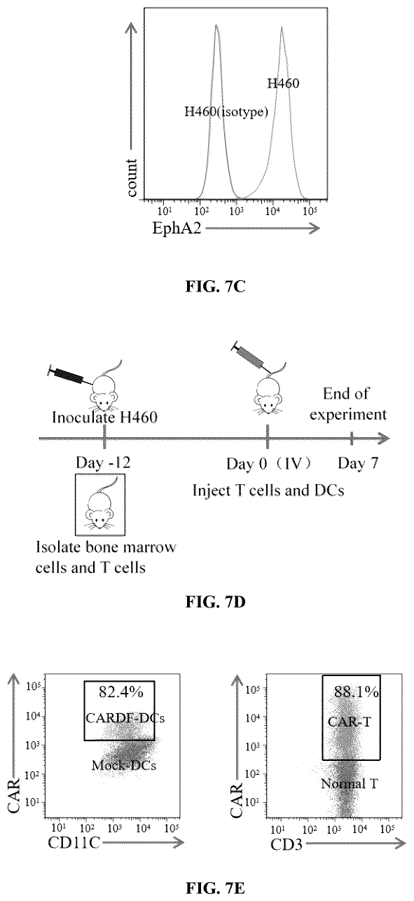

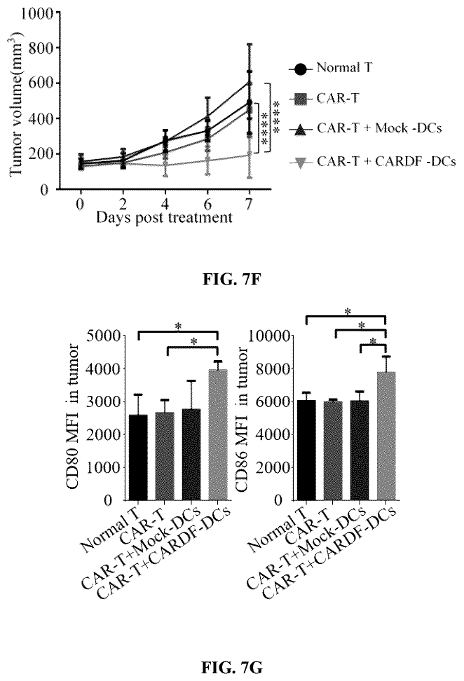

Solid tumors are heterogenous in the context of TIME (V Thorsson et al., The Immune Landscape of Cancer. Immunity 48, 812-830 e814 (2018)). To test the hypothesis that CARDF-DCs can reverse TIME of different solid tumors towards the pro-inflammatory conditions, we employed another human lung cancer cell line H460, which expressed higher levels of PD-L1 and also formed clinically relevant TIME in Hu-mice (FIGS. 7, A and B). The tumor cells were confirmed to express EphA2 (FIG. 7C). Consistent with the findings in lung tumors formed by A549 in Hu-mice, CARDF-DCs efficiently rescued the anti-tumor activities of CAR-T cells and suppress solid tumors formed by H460 cells in HuS-mice (FIG. 7, D to F). CARDF-DCs could infiltrate H460 tumors more efficiently or survived longer than control DCs (FIG. 7G). Therefore, these data reveal the uniform T cell activating capability of CARDF-DCs to reverse immune suppression in heterogenous TIME of solid tumors.

### Example 7

### Discussion

Despite the outstanding efficacy of CAR-T cell therapy to treat blood malignancies, the immunotherapy of solid tumors remains challenging due to the presence of immune suppressive microenvironment (TIME). Therefore, it is critical to develop strategies to disrupt TIME in order to improve the efficacy of immunotherapy of solid tumors. It is well established that the immune suppressive TIDCs play a key role in establishing TIME by suppressing cytotoxic T cell functions and promoting the immune suppressing regulatory T cells (J. M Tran Janco et al., Tumor-infiltrating dendritic cells in cancer pathogenesis. J. Immunol. 194, 2985-2991 (2015)). To achieve this goal, we developed a CAR-DC strategy that allow DCs to specifically target tumor cells and remain activated after encountering TIME. In this context, we show that the standard CAR for T cells fails to activate DCs after DCs encounter TIME. In order to specifically activate DCs, we designed DC activating CAR molecules with the intracellular domain composed of various DC-activating domains. After testing CARs with various combinations of DC activating domains, we discover that the tandem ligation of the cytoplasmic tails of Dectin1 and FcRγ can effectively activate DCs after they encounter TIME.

One of the key bottlenecks for tumor immunotherapy research is the lack of clinically relevant in vivo models to evaluate the efficacy of immunotherapies (P. S. Hegde et al., Top 10 Challenges in Cancer Immunotherapy. Immunity 52, 17-35 (2020)). For example, the solid tumors established in the immunodeficient mice failed to develop TIME, enabling efficient elimination of solid tumors by CAR-T cells in this model. To resolve this bottleneck, we developed two humanized mouse models that develop human solid tumors with clinically relevant TIME. First, the formation of solid tumors by CTLA4-Ig/PD-L1 overexpressing human tumor cells in immunodeficient mice developed an immune suppressive microenvironment. Second, to recapitulate the heterogenicity of TIME of solid tumors, the formation of solid tumors by human tumor cells in the immune system humanized mice develop clinically relevant TIME. Using these models, we demonstrate that human CAR-DCs can promote CAR-T cells to suppress solid tumors harboring clinically relevant TIME. In this context, this is the first report to show that CAR-DCs can reverse TIME towards pro-inflammatory conditions and activate the antitumor activities of CAR-T cells to suppress solid tumors.

Considering the heterogenicity of solid tumors, it will be important to examine whether CAR-DCs can reverse TIME of various types of human solid tumors. In addition, one potential limitation of this strategy is that cancer patients might not have sufficient and healthy DCs remaining after multiple rounds of chemotherapy or radiotherapy. This problem could be mitigated by recent progress to derive functional DCs from patient's induced pluripotent stem cells (D. Todorova et al., hESC-derived immune suppressive dendritic cells induce immune tolerance of parental hESC-derived allografts. E Bio Medicine 62, 103120 (2020); S. Senju et al., Generation of dendritic cells and macrophages from human induced pluripotent stem cells aiming at cell therapy. Gene Ther. 18, 874-883 (2011); S. Sontag et al., Modelling IRF8 Deficient Human Hematopoiesis and Dendritic Cell Development with Engineered iPS Cells. Stem cells 35, 898-908 (2017)). Based on the capability of CAR-DCs to reverse the immune suppressive TIME to the pro-inflammatory state, it will be interesting to examine the combination of CAR-DCs with other immunotherapies to treat malignant solid tumors. For example, CAR-DCs might promote the anti-tumor activity of Natural Killer cells and the immune checkpoint inhibitors such as anti-PD1 antibody, which are only effective for a small fraction of solid tumors (T Walzer et al., Natural-killer cells and dendritic cells: “l'union fait la force”. Blood 106, 2252-2258 (2005); E. Mamessier et al., Human breast cancer cells enhance self tolerance by promoting evasion from NK cell antitumor immunity. J. Clin. Invest. 121, 3609-3622 (2011); K. Foley et al., Current progress in immunotherapy for pancreatic cancer. Cancer lett. 381, 244-251 (2016); J. S. O'Donnell et al., Resistance to PD1/PDL1 checkpoint inhibition. Cancer Treat. Rev. 52, 71-81 (2017)67-70). The new humanized solid tumor models with clinically relevant TIME used here will provide ideal platforms to evaluate the efficacy of these combinational immunotherapies. In summary, the CAR-DC approach represents a promising and potentially universal strategy to overcome TIME to dictate the outcome of the immunotherapies of malignant solid tumors.

### Example 8

### Materials and Methods

### Study Design

Results shown are mean values with standard derivation. The number of independent experimental repeats is indicated in the figure legends. For the in vivo experiments of tumor growth, the animals were divided blindly into treatment groups before treatment and measurement, e.g., tumor weight and volume measurements, RT-qPCR assays, flow cytometry analyses, or ELISA measurements. Primary data are included in data file S1.

### Animal Studies

NOD/SCID/IL−27−/− (NSG) mice were purchased from Nanjing Model biology Company. NSG mice and Hu-mice employed in this study were maintained in a pathogen-free barrier animal facility. All animal work was approved by Institutional Animal CARE and Use Committee (IACUC).

### Construction of Lentiviral Vector Containing Chimeric Antigen Receptor (CAR)

The structure of the 2nd generation CAR anti-CD19 and anti-EphA2 are composed of a CD8 leader sequence and scFv, a CD8 transmembrane domain, and a 4-1BB and CD3ξ intracellular domain. To generate the DC CAR, the intra-cytoplasmic sequence of TLR4 (NM_138554.5), TNFR2 (NM_001066.3) as well as Dectin1 (NM_197947), FcRγ(NM_004106) were amplified to replace the regions of 4-1BB and CD3ξ intracellular domains within 2nd CAR. All sequences were optimized and synthesized by IGene company (Guangzhou). The expression cassettes were then cloned into the lenti-Cas9 vector (Addgene) by replacing Cas9 region.

### Primary Cells and Cell Lines Culture

DCs were generated from monocytes isolated from PBMC (LDEBIO Cat #1501). Briefly, the monocytes were isolated by anti-CD14 microbeads (Miltenyi Biotech Cat #130-050-201) and an autoMACS Pro separator apparatus. The monocytes were then cultured with GM-CSF (100 ng/ml; PeproTech Cat #300-03) and IL-4 (100 ng/ml; PeproTech Cat #200-04) in RPMI 1640 (Corning) supplied with 10% FBS (Gibco), 100 units/ml penicillin and 100 g/ml streptomycin (Thermo Fisher Scientific) for 5-6 days to generate immature DCs. Cytokines were replenished every 2-3 days. The maturation of DCs was performed for 24 hours with TNF-α (10 ng/ml; PeproTech Cat #300-01A) and LPS (3 g/ml; Sigma-Aldrich Cat #L4391).

Primary T cells were isolated by anti-CD3 microbeads (Miltenyi Biotech Cat #130-050-101) from the peripheral blood mononuclear cells and maintained in RPMI 1640 complemented with 10% FBS, 2 mM L-glutamine (Thermo Fisher Scientific), 1% penicillin-streptomycin, 2-mercaptoethanol (25 μM, Gibco) and 100 U/ml human IL-2 (PeproTech Cat #AF-200-02-500).

Both A549 (Cat #ATCC® CCL-185™) and H460 (Cat #ATCC® HTB-177™) were purchased from ATCC, Manassas, VA. A549-CP construction was performed as we previously described (60). H460-CD19 was constructed by overexpressing human CD19 on surface of H460 using lentivirus. THP-1 cell line (Cat #ATCC® TIB-202™) is a leukemia cell line established from a patient with chronic myelogenous leukemia. All above cells were cultured in RPMI 1640 complemented with 10% FBS, 2 mM L-glutamine, 1% penicillin-streptomycin, and 25 μM 2-mercaptoethanol. 293FT cells (Thermo scientific Cat #R70007) were cultured in Dulbecco's Modified Eagle's medium (DMEM, Thermo Fisher Scientific) complemented with 10% FBS and 1% penicillin-streptomycin. Above cell lines were passaged using 0.25% Trypsin-EDTA (Thermo Fisher Scientific) at appropriate ratios when cells reaching full confluence. All cells were incubated in a dark humidity 37° C. incubator with 5% CO2.

### Transduction of Mo-DCs

Human monocytes were transferred to 24-well ultra-low attachment tissue culture plates at a density of 2-5×105 cells per well/400 uL differentiation media (RPMI1640 complete media supplied with 100 ng/ml GM-CSF and 100 ng/ml IL-4) prior to transduction. Lentivirus quantity was calculated by qPCR Lentivirus Titration (Titer) Kit (ABM Cat #LV900-iC). Transduction was performed using MOI 100 by thawing the titrated lentivirus stocks at 37° C. Mixing the appropriate volume of virus concentrate with 6 ug/ml Protamine Sulfate (Sigma-Aldrich Cat #1578612-2) in differentiation media to achieve a total volume of 500 ul per well. After 12 hours incubation at 37° C., additional 500 ul of differentiation media was added to each well. Most of the culture media was aspirated at 24 h post-transduction, cells were washed twice with PBS, and further cultured in differentiation media. On day 5, Mo-DCs were collected for future co-culture experiments or directly matured by TNF-α (10 ng/ml; PeproTech) and LPS (3 g/ml; Sigma-Aldrich) for 24-48 hours.

### Transduction of iPSCs

The engineered cell (e.g., CAR-DCs) of the present disclosure can also be prepared by transfecting a viral vector (e.g., lentiviral vector) provided herein to human induced pluripotent stem cells (hiPSCs) to prepare stable CAR expressing cell lines (such as CARDF-hiPSCs). The hiPSCs have the ability to proliferate immortally and differentiate into various tissue cells and have great potential in the cell therapy of diseases. In the present disclosure, the OP9 stromal cell nutrition method (Nat Protoc. 2011 March; 6(3): 296-313. doi: 10.1038 nprot. 2010.184) is preferably used to induce the differentiation of hiPSC into DC. In the present disclosure, the initial number of differentiated cells from hiPSCs is preferably 1×10⁶ to 1.5×10⁶, and the initial medium is preferably a complete medium of MEM-α supplemented with 20% fetal bovine serum and 1% penicillin-streptomycin. The entire process of DC cell differentiation preferably takes about 31 to 38 days. The CARDF-hiPSCs of the present disclosure can be induced to differentiation on a large scale to produce homogeneous CARDF-DCs. The CARDF-DCs derived the hiPSCs are expected to have functions, such as disrupting TIME, converting TIME into an inflammatory state, capable of activating DCs in an immune suppressive tumor microenvironment, etc., as described above.

### Preparation of CAR-T Cells

Primary CD3+ T cells were isolated from PBMCs and activated with Human T cell activation Kit following the manufacturer's instructions. Briefly, 12-well plate was coated with 3 ug/mL PBS-diluted anti-CD3 antibody (BD Cat #555329; RRID:AB_395736) overnight at 4° C., and the plate was washed twice with PBS the next day, then T cells were thawed and transferred into the plate with 1 ug/mL anti-CD28 antibody (BD Cat #555725; RRID:AB_396068) in T cell media. Activation lasted for two days, and at the third day activated T cells were harvested and infected with lentivirus expressing indicated T-CAR construct. In brief, T cells were transferred to 24-well tissue culture plates at a density of 5×10⁵ cells per well/400 uL T cell media prior to transduction. Transductions were performed using MOI 10 by thawing the titrated virus stocks at 37° C. Mixing the appropriate volume of virus concentrate with 10 ug/ml polybrene (Sigma-Aldrich Cat #TR-1003-G) in media to achieve a total volume of 500 ul per well. After 12 hours incubation at 37° C., additional 500 ul of media was added to each well. At 24 h post-transduction, cells were collected, washed with PBS twice and re-suspended in T cell media, continuing to culture for proliferation. On the day of killing assays (almost day 10 post activation), cells were collected and analyzed by flow cytometry, and cells counts were obtained using a haemocytometer.

### In Vitro T Cells Proliferation Assays

Primary CD3+ T cells were stained with CellTrace-CFSE (Life Technologies Cat #65-0850-84) following the manufacturer's instructions. In the experiment, DCs were pre-incubated for 48 hours with cancer targets (H460-CD19 cells, A549 cells or A549-CP cells) at 1:1 ratio in 48-well plates, and then primary T cells (DCs:T cells=1:5) were added to the co-culture. In other experiments, DCs and T cells were incubated with cancer targets concomitantly (day 0). Unless specifically indicated, ratio of targets: DCs:T cells=1:1:5 was performed in each cell co-culture condition. The proliferation was analyzed using flow cytometry by gating the Live CD3+ T cells.

### In Vitro DCs and Tumor Cells Co-Culture Assays

1×106 H460-CD19 cells, A549 cells or A549-CP cells were co-cultured with 1×106 Mock-DCs or CAR-DCs derived from THP-1 or monocytes in 6-well plates, 48 hours post co-culture, cells were treated with 0.25% Trypsin-EDTA for 5 min at 37° C., washed with PBS, then the cells were stained with directly Fluorochrome-conjugated antibodies CD11C, CD80, CD86, HLA-ABC, HLA-DR and analyzed by flow cytometry.

### In Vitro Killing Assays

### CD19 Targets

Approximately 1×10⁴ H460 cells and 1×10⁴ H460-CD19 cells (target cells) were plated in 200 ul RPMI1640 complete media in each well of 48-well-plate, 2×10⁴ WT-DCs or CARDF-DCs (stimulator cells) in 100 ul RPMI1640 media were added to corresponding wells, 10⁵ CAR-T cells (effector cells) in 100 ul RPMI1640 media were added to corresponding wells. Continue complementing media to 400 ul in the deficient wells. After incubating for 24 hours, the remaining cells were collected for flow cytometry and culture supernatant for subsequent assays. Percentage of specific cytolysis was calculated for each well as follows: % of specific lysis=(% CD19 (tumor cells only)−% CD19 (killing group))/CD19% (tumor cells only)×100%.

### EphA2 Targets

Approximately 2×10⁴ A549 cells or 2×10⁴ A549-CP cells (target cells) were plated in 200 ul RPMI1640 complete media in each well of 48-well-plate, 2×10⁴ Mock-DCs or CAR-DCs (stimulator cells) in 100 ul RPMI1640 media were added to corresponding wells, 1×10⁵ CAR-T cells (effector cells) in 100 ul RPMI1640 media were added to corresponding wells. Continue complementing media to 400 ul in the deficient wells. After incubating for 12 hours, 24 hours, remaining cells were collected for flow cytometry and culture supernatant for subsequent assays.

### IFN-γ Staining

After in vitro killing assays of A549-CP tumor cells, residual cells were collected and stained using Intracellular staining kit (BD Biosciences) following the manufacturer's instructions. Briefly, fixing and permeabilizing the cells with 200 ul fixation/permeabilization buffer for 20 minutes on ice, then washing twice with 1× wash buffer. Cells were stained with IFN-γ-BV650, CD3-V450, CD8-PE for 30 min at 4° C. in wash buffer, then washed twice with 1× wash buffer before flow cytometry analysis.

### IFN-γ and LDH Assays

Culture supernatant from in vitro killing assays was collected and tested for cytokine IFN-γ levels by ELISA Kit (Invitrogen Cat #88-7316-76), and LDH levels by CytoTox96® Non-Radioactive Cytotoxicity Assay (Promega Cat #G1780) following the manufacturer's instructions. The supernatant was diluted to 1:50 or 1:100 according to the preliminary experiments.

### Tumor Xenograft Model in NSG Mice Study

A549WT and A549-CP tumor models were generated by injecting 1.5×10⁶ cells in 100 μl of PBS subcutaneously into the both flanks of 6-week-old NSG mice. In the experiments, T cells and DCs were infused on day 5 and 14 post-tumor challenge via intravenous injection of 5×10⁶ DCs and 1×10⁷ CAR-T cells in 500 μl of PBS. The volume of tumors was determined by caliper measurements and calculated using the formula: volume (mm³)=½×D×d2, which D is the longer and d the shorter tumor axis. When the mice were administered euthanasia, all tumors were collected, weighted, and photographed. Besides, mice spleens and blood were collected, separated and processed into single cells, stained with indicated fluorochrome-conjugated antibodies and analyzed by flow cytometry.

### Tumor Xenograft Model in Hu-Mice Study

Detailed description of Hu-mice generation can be found in, for example, Rong Z et al., An Effective Approach to Prevent Immune Rejection of Human ESC-Derived Allografts. Cell Stem Cell 14, 121-130 (2014). Generation of Hu-mice derived DCs were differentiated from bone marrow cells according to published protocols. Briefly, femurs and tibias of Hu-mice were removed with sterile scissors, immersed in 70% alcohol for 3 minutes, and washed twice with ice-cold PBS. Then the marrow cells were flushed out using a sterile syringe (26 gauge needle). The marrow cells were re-suspended, passed through 70 m nylon mesh, then erythrocyte was lysed with Lyse buffer (BD Bioscience). The remaining cells were washed twice with PBS and counted, 1×10⁶ cells/ml were adjusted with complete RPMI-1640 medium supplemented with 20 ng/ml human GM-CSF and 5 ng/ml human IL-4. Transfer 3 ml of cell suspension into each well of 6-well plate. The culture medium was changed every 2 days by gently swirling the plates, aspirating half of the medium, and adding fresh medium containing GM-CSF and IL-4. After 9 days of culture, cells were collected and washed, stained with anti-human CD11C antibody and analyzed by flow cytometry. For CARDF transduction, immature BM-DCs were transferred to 6-well tissue culture plates at a density of 50×10⁵-10×10⁵ cells per well/ml differentiation media (RPMI1640 complete media supplied with 20 ng/ml GM-CSF and 5 ng/ml IL-4) prior transduction. Transduction was performed using MOI 100 by thawing the titrated lentivirus stocks at 37° C. Mixing the virus concentrate with 6 ug/ml Protamine Sulfate in differentiation media. After 12 hours incubation at 37° C., additional 1 ml of differentiation media was added to each well. Most of the culture media was aspirated at 24 h post-transduction, cells were washed twice with PBS, and further cultured in differentiation media until use.

Generation of T cells from Hu-mice was isolated from splenic cells. Briefly, the spleen of Hu-mice was removed with sterile tweezers and immersed in ice-cold PBS for 3 minutes, then ground on the 70 m nylon mesh surface using the bottom of the syringe. Single cells were flushed through the mesh and washed with PBS. Then erythrocytes were lysed. T cells were separated by anti-human CD3 magnetic microbeads and then maintained in RPMI 1640 complete media supplied with 100 U/ml human IL-2. CAR-T cells were prepared as mentioned above.

1.5×10⁶ A549 cells in 100p of PBS supplied with Matrigel were inoculated subcutaneously into the both flanks of Hu-mice. 8 days later, the tumor-bearing Hu-mice were randomly distributed into four cohorts. In the experiments, DCs and T cells were infused via tail intravenous injection of 3×10⁶ DCs and 1×10⁷ CAR-T cells in 400 μl of PBS. 2×10⁶ H460 cells in 100p of PBS supplied with Matrigel were inoculated subcutaneously into the both flanks of Hu-mice. 13 days later, the tumor-bearing Hu-mice were randomly distributed into four cohorts. In the experiments, DCs and T cells were infused via tail intravenous injection of 3×10⁶ DCs and 1×10⁷ CAR-T cells in 400 μl of PBS. The volume of tumors was determined by caliper measurement and calculated using the formula: volume (mm3)=½×D×d2, which D is the longer and d the shorter tumor axis. When the mice were administered euthanasia, tumors, spleens, bone marrow and blood were collected for analyses.

### Tumor Tissue Digestion and Staining

The harvested paired tumors engrafted in one Hu-mice were mixed, cut into patches and dissociated using tissue digestive enzyme solution [100 Kunitz units of DNaseI (STEM CELL Cat #07900), 8 Wunsch units of Liberase™™ (Sigma Cat #LIB™-RO) (8 U/mL) and Liberase™ TH (Sigma Cat #LIBTH-RO) (8 U/mL) in medium 199 (GIBCO) with 20 μM HEPES (GIBCO)]. After shaking at 37° C., 150 rpm for 1.5 hours, the digestion was stopped by adding 5 mL RPMI-1640 containing 10% FBS. Subsequently, the suspension was filtered through 40 m Cell strainer (Corning) and cells acquired were subjected to antibody staining for flow cytometry analyses.

### Flow Cytometry Analyses

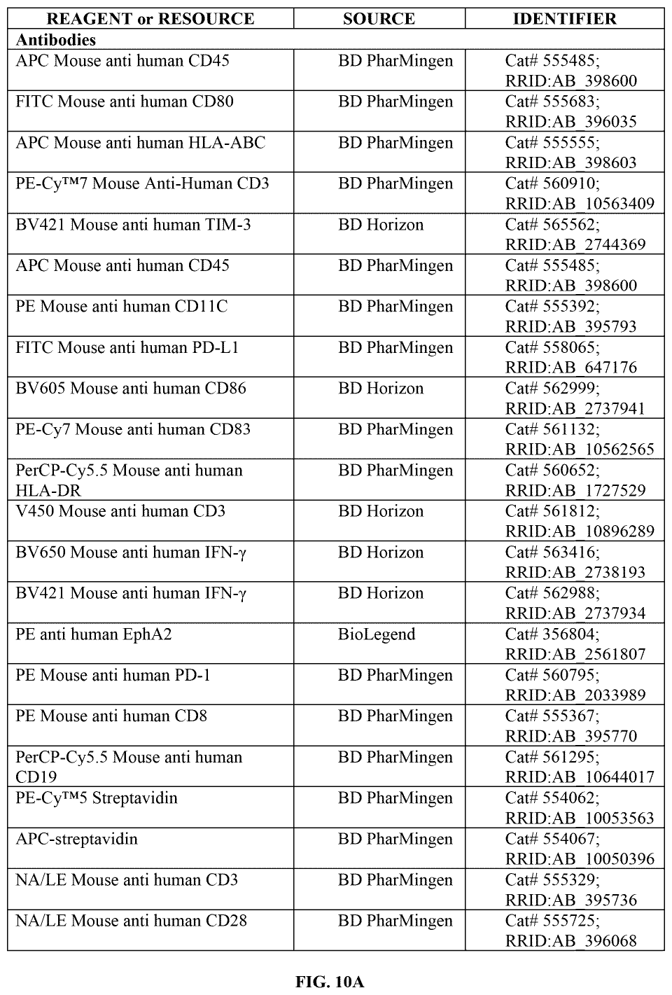

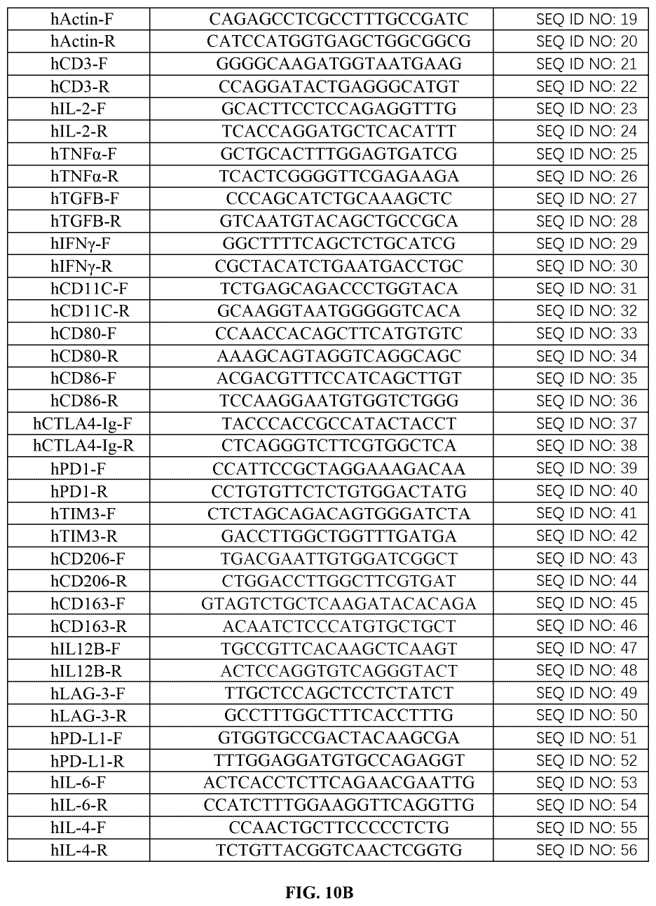

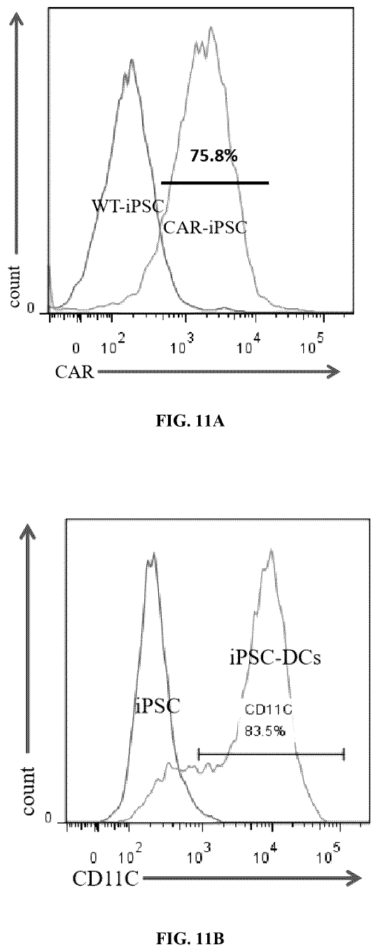

All flow cytometry analysis was performed by the LSR Fortessa (BD Biosciences). Flow data was analyzed using FlowJo software (Tree Star, Ashland, OR). Relevant sample gating has been provided in extended data figures. Fluorochrome-conjugated antibodies APC-CD45, PE-CD11C, FITC-CD80, BV605-CD86, PE-cy7-CD83, APC-HLA-ABC, BV510-HLA-DR, V450-CD3, PE-cy7-CD3, BV421-TIM-3, PE-PD-1, PE-CD8, BV650-IFNγ, BV421-IFNγ, FITC-PDL1, Percp-cy5.5-CD19, PE-cy5-streptavidin, APC-streptavidin were purchased from BD Sciences, FITC-TIM-3 was purchased from Miltenyi Biotech, Biotin-Protein L was purchased from GenScript, PE-EphA2 was purchased from BioLegend. For dendritic cells staining assay, FcR blocking reagent (Miltenyi Biotech) was used following the manufacturer's instructions. For surface markers staining, cells were spun down and stained with diluted antibodies following the manufacturer's instructions, in FACS Buffer (PBS+1% FBS+2 mM EDTA) for 30 min at 4° C., then washed twice with PBS and immediately analyzed by flow cytometry. The Protein L staining needed the secondary antibody staining according to manufacturer's instructions. Please refer to FIG. 10A for antibody details.

### Statistical Analyses

Statistical analyses were performed using appropriate statistical comparisons, including unpaired two-tailed t-tests with Welch's correction, one-way ANOVA with Tukey's multiple comparisons test, two-way ANOVA followed by Tukey's multiple comparisons test as needed by Prism7 (GraphPad Software). Data were presented as mean±SD. P<0.05 was considered to be statistically significant.

### Example 9

### Supplementary Materials

### Materials and Methods

### THP-1 Cells Transduction and Differentiation into DCs

THP-1 cells were transferred to 24-well tissue culture plates at a density of 5×10⁵ cells per well/400 uL RPMI1640 complete media prior to transduction. Transduction was performed using MOI 10 by thawing the titrated lentivirus stocks at 37° C. Mixing the appropriate volume of virus concentrate with 6 ug/ml Protamine Sulfate in RPMI1640 complete media to achieve a total volume of 500 ul per well. After 12 hours incubation at 37° C., additional 500 ul media was added to each well. Most of the culture media was aspirated at 24 h post-transduction, cells were washed twice with PBS and further cultured. On day 3, cells were collected and transduction efficiency was analyzed by flow cytometry.

THP-1 or CAR+ THP-1 cells were harvested and resuspended in RPMI1640 complete media at a density of 2×10⁵ cells/ml, then every 3 ml cells suspension were transferred into one well of 6-well-plate. Recombinant human GM-CSF (100 ng/ml) and recombinant human IL-4 (100 ng/ml) are included in the culture media to stimulate DCs differentiation. Culture media was exchanged every 2 or 3 days with fresh cytokine-supplemented media. DCs differentiation in the presence of cytokines lasted for at least 7-10 days before further experiments.

### Lentivirus Production

Plasmid DNA for lentivirus packaging was purified with NucleoBond Xtra Midi EF kits (Takara Bio Cat #740420.50) according to the manufacturer's instructions. PEI packaging method was conducted according to Addgene's lentivirus production protocol with minor modifications. Briefly, 293FT packaging cells were plated into 15 cm dishes at a 1:3 dilution ratio, next day when the confluence reaching 90%, media was changed 1 h before transfection, two packaging plasmids psPAX2 (Addgene Cat #12260) and pMD2.G (Addgene Cat #12259) together with the target plasmids were diluted in Opti-MEM (Gibco) with 1 mg/ml PEI at the DNA:PEI ratio of 1:3-1:4. After 20 min incubation at room temperature, plasmid mixtures were added into cells gently and media was replaced with complete DMEM media 8 hours after transfection. Lentivirus particles were harvested 48-72 hours post transfection using Lenti-X concentrator (Takara Bio Cat #631232) according to the manufacturer's instructions. Briefly, collected media was centrifuged at 1500 g for 15 min and supernatant was incubated with ⅓ volume of Lenti-X Concentrator overnight at 4° C. After centrifugation at 3000 rpm for 45 min at 4° C., viral pellets were resuspended in 0.6-0.8 ml cold PBS, aliquoted and stored at −80° C.

### Quantitative PCR Analyses

Total RNA was extracted from cells or tumor tissues using Trizol reagent (TaKaRa) as previously described. The cDNA was synthesized from 1 g total RNA using PrimeScript RT reagent kit (TaKaRa Cat #RR047A) following the manufacturer's instructions. Real time PCR analysis was performed using StepOnePlus Real-Time PCR System (Applied Biosystems) and Roche System (Lifescience) with TB Green reagent (TaKaRa Cat #RR820A) following the manufacturer's instructions. Primers sequences are shown in FIG. 10B.

TABLE 1
Sequences mentioned in the present disclosure

| SEQ | | |
|---|---|---|
| ID | | |
| NO: | Amino acid sequence/Nucleic acid sequence | Name |
| 1 | RWPPSAACSGKESVVAIRTNSQSDFHLQTYGDE | Cytoplasmic domain of |
| | DLNELDPHYEM | Dectin1 (bolded and |
| | | underlined sequence is |
| | | ITAM) |
| 2 | RLKIQVRKAAITSYEKSDGVYTGLSTRNQETYE | ITAM of FcγR (bolded |
| | TLKHEKPPQ | and underlined sequences |
| | | are ITAMs) |
| 3 | RWPPSAACSGKESVVAIRTNSQSDFHLQTYGDE | tandem amino acid |
| | DLNELDPHYEMRLKIQVRKAAITSYEKSDGVYT | sequence of the |
| | GLSTRNQETYETLKHEKPPQ | Cytoplasmic domains of |
| | | Dectin1 and FcγR |
| 4 | CGCTGGCCTCCTTCTGCAGCTTGTTCGGGAAA | tandem nucleic acid |
| | AGAGTCAGTTGTTGCTATAAGGACCAATAGCC | sequence of the |
| | AATCTGACTTCCACTTACAAACTTATGGAGAT | Cytoplasmic domains of |
| | GAAGATTTGAATGAATTAGATCCTCATTATGA | Dectin1 and FcγR |
| | AATGCGACTGAAGATCCAAGTGCGAAAGGCA | |
| | GCTATAACCAGCTATGAGAAATCAGATGGTGT | |
| | TTACACGGGCCTGAGCACCAGGAACCAGGAG | |
| | ACTTACGAGACTCTGAAGCATGAGAAACCACC | |
| | ACAG | |
| 5 | MALPVTALLLPLALLLHAARP | signal peptide of CD8 |
| | | alpha |
| 6 | IYIWAPLAGTCGVLLLSLVITLYC | transmembrane domain |
| | | of CD8 alpha |
| 7 | TTTPAPRPPTPAPTIASQPLSLRPEACRPAAGGAV | hinge region of CD8 alpha |
| | HTRGLDFACD | |
| 8 | MGVLLTQRTLLSLVLALLFPSMASMAMHVAQP | CTLA-4-Ig |
| | AVVLASSRGIASFVCEYASPGKATEVRVTVLRQ | |
| | ADSQVTEVCAATYMMGNELTFLDDSICTGTSSG | |
| | NQVNLTIQGLRAMDTGLYICKVELMYPPPYYLG | |
| | IGNGTQIYVIDPEPCPDSDQEPKSSDKTHTSPPSP | |
| | APELLGGSSVFLFPPKPKDTLMISRTPEVTCVVV | |
| | DVSHEDPEVKFNWYVDGVEVHNAKTKPREEQY | |
| | NSTYRVVSVLTVLHQDWLNGKEYKCKVSNKAL | |
| | PAPIEKTISKAKGQPREPQVYTLPPSRDELTKNQ | |
| | VSLTCLVKGFYPSDIAVEWESNGQPENNYKTTPP | |
| | VLDSDGSFFLYSKLTVDKSRWQQGNVFSCSVM | |
| | HEALHNHYTQKSLSLSPGK | |
| 9 | MRIFAVFIFMTYWHLLNAFTVTVPKDLYVVEYG | PD-L1 |
| | SNMTIECKFPVEKQLDLAALIVYWEMEDKNIIQF | |
| | VHGEEDLKVQHSSYRQRARLLKDQLSLGNAAL | |
| | QITDVKLQDAGVYRCMISYGGADYKRITVKVN | |
| | APYNKINQRILVVDPVTSEHELTCQAEGYPKAEV | |
| | IWTSSDHQVLSGKTTTTNSKREEKLFNVTSTLRI | |
| | NTTTNEIFYCTFRRLDPEENHTAELVIPELPLAHP | |
| | PNERTHLVILGAILLCLGVALTFIFRLRKGRMMD | |
| | VKKCGIQDTNSKKQSDTHLEET | |
| 10 | GFTFSSYTMS | HCDR1 of scFv for |
| | | EphA2 |
| 11 | TISSRGTYTYY PDSVKG | HCDR2 of scFv for |
| | | EphA2 |
| 12 | EAIFTH | HCDR3 of scFv for |
| | | EphA2 |
| 13 | KASQDINNYHS | LCDR1 of scFv for |
| | | EphA2 |
| 14 | RANRLVD | LCDR2 of scFv for |
| | | EphA2 |
| 15 | LKYNVFPYT | LCDR3 of scFv for |
| | | EphA2 |
| 16 | QVQLLESGGGLVQPGGSLRLSCAASGFTFSSYT | VH of scFv for EphA2 |
| | MSWVRQAPGQALEWMGTISSRGTYTYYPDSVK | |
| | GRFTISRDNAKNSLYLQMNSLRAEDTAVYYCAR | |
| | EAIFTHWGRGTLVTVSS | |
| 17 | DIQLTQSPSSLSASVGDRVTITCKASQDINNYHS | VL of scFv for EphA2 |
| | WYQQKPGQAPRLLIYRANRLVDGVPDRFSGSGY | |
| | GTDFTLTINNIESEDAAYYFCLKYNVFPYTFGQG | |
| | TKVEIK | |
| 18 | QVQLLESGGGLVQPGGSLRLSCAASGFTFSSYT | full length of scFv for |
| | MSWVRQAPGQALEWMGTISSRGTYTYYPDSVK | EphA2 |
| | GRFTISRDNAKNSLYLQMNSLRAEDTAVYYCAR | |
| | EAIFTHWGRGTLVTVSSGGGGSGGGGSGGGGSD | |
| | IQLTQSPSSLSASVGDRVTITCKASQDINNYHSW | |
| | YQQKPGQAPRLLIYRANRLVDGVPDRFSGSGYG | |
| | TDFTLTINNIESEDAAYYFCLKYNVFPYTFGQGT | |
| | KVEIK | |
| 19 | CAGAGCCTCGCCTTTGCCGATC | hActin-F |
| 20 | CATCCATGGTGAGCTGGCGGCG | hActin-R |
| 21 | GGGGCAAGATGGTAATGAAG | hCD3-F |
| 22 | CCAGGATACTGAGGGCATGT | hCD3-R |
| 23 | GCACTTCCTCCAGAGGTTTG | hIL-2-F |
| 24 | TCACCAGGATGCTCACATTT | hIL-2-R |
| 25 | GCTGCACTTTGGAGTGATCG | hTNFα-F |
| 26 | TCACTCGGGGTTCGAGAAGA | hTNFα-R |
| 27 | CCCAGCATCTGCAAAGCTC | hTGFB-F |
| 28 | GTCAATGTACAGCTGCCGCA | hTGFB-R |
| 29 | GGCTTTTCAGCTCTGCATCG | hIFNγ-F |
| 30 | CGCTACATCTGAATGACCTGC | hIFNγ-R |
| 31 | TCTGAGCAGACCCTGGTACA | hCD11C-F |
| 32 | GCAAGGTAATGGGGGTCACA | hCD11C-R |
| 33 | CCAACCACAGCTTCATGTGTC | hCD80-F |
| 34 | AAAGCAGTAGGTCAGGCAGC | hCD80-R |
| 35 | ACGACGTTTCCATCAGCTTGT | hCD86-F |
| 36 | TCCAAGGAATGTGGTCTGGG | hCD86-R |
| 37 | TACCCACCGCCATACTACCT | hCTLA4-Ig-F |
| 38 | CTCAGGGTCTTCGTGGCTCA | hCTLA4-Ig-R |
| 39 | CCATTCCGCTAGGAAAGACAA | hPD1-F |
| 40 | CCTGTGTTCTCTGTGGACTATG | hPD1-R |
| 41 | CTCTAGCAGACAGTGGGATCTA | hTIM3-F |
| 42 | GACCTTGGCTGGTTTGATGA | hTIM3-R |
| 43 | TGACGAATTGTGGATCGGCT | hCD206-F |
| 44 | CTGGACCTTGGCTTCGTGAT | hCD206-R |
| 45 | GTAGTCTGCTCAAGATACACAGA | hCD163-F |
| 46 | ACAATCTCCCATGTGCTGCT | hCD163-R |
| 47 | TGCCGTTCACAAGCTCAAGT | hIL12B-F |
| 48 | ACTCCAGGTGTCAGGGTACT | hIL12B-R |
| 49 | TTGCTCCAGCTCCTCTATCT | hLAG-3-F |
| 50 | GCCTTTGGCTTTCACCTTTG | hLAG-3-R |
| 51 | GTGGTGCCGACTACAAGCGA | hPD-L1-F |
| 52 | TTTGGAGGATGTGCCAGAGGT | hPD-L1-R |
| 53 | ACTCACCTCTTCAGAACGAATTG | hIL-6-F |
| 54 | CCATCTTTGGAAGGTTCAGGTTG | hIL-6-R |
| 55 | CCAACTGCTTCCCCCTCTG | hIL-4-F |
| 56 | TCTGTTACGGTCAACTCGGTG | hIL-4-R |
| 57 | GGGGS GGGGSGGGGS | linker |
| 58 | YGDEDL | ITAM of Dectin1 |
| 59 | YTGL | ITAM1 of FcγR |
| 60 | YETL | ITAM2 of FcγR |

1-78. (canceled)

79. A polynucleotide encoding a chimeric antigen receptor (CAR), wherein the CAR comprising (1) an extracellular antigen-binding domain, (2) a transmembrane domain and (3) an intracellular signaling domain,
wherein the intracellular signaling domain comprises a cytoplasmic domain of a dendritic cell activating receptor selected from the group consisting of RIG-1, NLRP10, DEC-205, BDCA-2, CD86, 4-1BBL, OX40L, CD40, IFNAR, TLR4, TNFR (e.g., TNFR2), CD80, CD40L, CD367 (DCIR), CD207 (Langerin), CD371 (DCAL-2, CLEC12a), CD204, CD36, IFNγR, Dectin-1 and FcγR, or a combination thereof,
wherein the CAR is capable of activating dendritic cells in an immune suppressive tumor microenvironment,
wherein the polynucleotide is a DNA or RNA.

80. The polynucleotide of claim 79, wherein the immune suppressive tumor microenvironment comprises a tumor that has poor responsiveness to monotherapy of adoptive cell therapy (e.g., CAR-T monotherapy) or a tumor and/or tumor infiltrating immune cells that are: 1) expressing an immune inhibitory molecule, and/or 2) deficient in an immune stimulating cytokine,
wherein the immune inhibitory molecule is selected from the group consisting of PD-1, TIM-3, TIGIT, LAG-3, A2AR, BTLA (CD272), CTLA-4 (CD152), IDO1, IDO2, TDO, NOX2, VISTA, SIGLEC7 (CD328), PVR(CD155) and SIGLEC9 (CD329), PD-L1, PD-L2, B7-H3 (CD276), B7-H4 (VTCN1), PVR(CD155), HLA class I, sialoglycoprotein, CD112, CD113, Galectin9, CD24, and CD47,
wherein the immune stimulating cytokine is selected from TNF-a, IFN-β, IFN-γ, IL-1, IL-2, IL-4, IL-6, IL-8, IL-10, IL-12, IL-18, granulocyte-macrophage colony stimulating factor and a combination thereof.

81. The polynucleotide of claim 79, wherein the intracellular signaling domain comprises the cytoplasmic domain of Dectin-1 and the cytoplasmic domain of FcγR.

82. The polynucleotide of claim 81, wherein the cytoplasmic domain of Dectin-1 comprises an amino acid sequence set forth in SEQ ID NO: 1, or any functional forms thereof, or the cytoplasmic domain of FcγR comprises an amino acid sequence set forth in SEQ ID NO: 2 or any functional forms thereof.

83. The polynucleotide of claim 81, wherein the intracellular signaling domain comprises an amino acid sequence set forth in SEQ ID NO: 3 or any functional forms thereof or an amino acid sequence encoded by a nucleic acid sequence set forth in SEQ ID NO: 4 or any functional forms thereof.

84. The polynucleotide of claim 79, wherein the extracellular antigen-binding domain comprises a single-chain variable fragment (scFv), the scFv is specific for a tumor surface marker (e.g., solid tumor surface marker).

85. The polynucleotide of claim 84, wherein the tumor surface marker is selected from the group consisting of: EphA2, CD19, CD70, CD133, CD147, CD171, DLL3, EGFRvIII, Mesothelin, ganglioside GD2, FAP (fibroblast activating protein), FBP (folate binding protein), Lewis Y, Claudin 18.2, IL13Rα2, HER2, MDC1, PMSA (prostate membrane specific antigen), ROR1, B7-H3, CAIX, CD133, CD171, CEA, GPC3, MUC1, NKG2D.

86. The polynucleotide of claim 79, wherein the CAR further comprises a signal peptide, the signal peptide comprises a signal peptide of CD8 alpha.

87. The polynucleotide of claim 86, wherein the signal peptide of CD8 alpha comprises a sequence set forth in SEQ ID NO: 5 or any functional forms thereof.

88. The polynucleotide of claim 79, wherein the transmembrane domain comprises a transmembrane domain of CD8 alpha.

89. The polynucleotide of claim 88, wherein the transmembrane domain of CD8 alpha comprises a sequence set forth in SEQ ID NO: 6, or any functional forms thereof.

90. The polynucleotide of claim 79, wherein the extracellular antigen-binding domain is linked to the transmembrane domain by a hinge region, the hinge region comprises a hinge region of CD8 alpha.

91. The polynucleotide of claim 90, wherein the hinge region of CD8 alpha comprises a sequence set forth in SEQ ID NO: 7, or any functional forms thereof.

92. A polypeptide encoded by the polynucleotide of claim 79.

93. A vector comprising the polynucleotide of claim 79, wherein the polynucleotide encoding the CAR is operably linked to at least one regulatory polynucleotide element for expression of the CAR.

94. An engineered cell comprising the polypeptide of claim 92.

95. A method of producing one or more engineered cells, each comprising a polypeptide encoded by the polynucleotide of claim 79, the method comprising introducing to a starting cell the vector comprising the polynucleotide of claim 79, wherein the polynucleotide encoding the CAR is operably linked to at least one regulatory polynucleotide element for expression of the CAR, under conditions suitable for expression of the polynucleotide of claim 79.

96. The method of claim 95, wherein the starting cell is a dendritic cell or a precursor or a progenitor cell thereof which is derived from a peripheral blood cell, a bone marrow cell, an embryonic stem cell, or an induced pluripotent stem cell.

97. A population of cells produced ex vivo by the method of claim 95.

98. A pharmaceutical composition comprising:
(i) the polynucleotide of claim 79, or
a polypeptide encoded by the polynucleotide of claim 79, or
a vector comprising the polynucleotide of claim 79, wherein the polynucleotide encoding the CAR is operably linked to at least one regulatory polynucleotide element for expression of the CAR, or
a population of engineered cells, each comprising a polypeptide encoded by the polynucleotide of claim 79, or
a population of cells produced ex vivo by a method of producing one or more engineered cells, each comprising a polypeptide encoded by the polynucleotide of claim 79, the method comprising introducing to a starting cell the vector comprising the polynucleotide of claim 79, wherein the polynucleotide encoding the CAR is operably linked to at least one regulatory polynucleotide element for expression of the CAR, under conditions suitable for expression of the polynucleotide of claim 79, and
(ii) a pharmaceutically acceptable medium.

99. A method for improving efficacy of adoptive cell therapy in treating cancer in a subject in need thereof, comprising administering a therapeutically effective amount of the pharmaceutical composition of claim 98.

100. A method of inducing proliferation of immune cells, prolonging the survival of immune cells, and/or increasing expression and/or secretion of immune stimulating cytokines from immune cells in an immune suppressive microenvironment, comprising contacting the immune suppressive microenvironment with the engineered cell of claim 94, wherein the immune cell is autologous or allogeneic.

101. A method of treating a disease or pathological condition in a subject in need thereof, comprising administering a therapeutically effective amount of the pharmaceutical composition of claim 98.

102. A method of selecting a CAR capable of activating dendritic cells, comprising:
(a) providing a non-human animal comprising an immune suppressive tumor microenvironment,
(b) administering a dendritic cell expressing a candidate CAR to the non-human animal,
c) detecting a marker for the dendritic cell activation selected from improved infiltration to the immune suppressive tumor microenvironment, improved survival rate, and enhanced function in inducing activation of an immune cell when compared to a reference dendritic cell, and
(d) selecting the candidate CAR as a CAR capable of activating dendric cells,
wherein the immune suppressive tumor microenvironment is clinically relevant, comprising a tumor and/or tumor infiltrating immune cells expressing an immune inhibitory molecule.

103. The method of claim 102, wherein the non-human animal comprises human fetal thymus and autologous human hematopoietic stem cells (e.g., human CD34+ hematopoietic stem cells).

104. The method of claim 102, wherein the immune inhibitory molecule is selected from the group consisting of PD-1, TIM-3, TIGIT, LAG-3, A2AR, BTLA (CD272), CTLA-4 (CD152), IDO1, IDO2, TDO, NOX2, VISTA, SIGLEC7 (CD328), PVR(CD155) and SIGLEC9 (CD329), PD-L1, PD-L2, B7-H3 (CD276), B7-H4 (VTCN1), PVR(CD155), HLA class I, sialoglycoprotein, CD112, CD113, Galectin9, CD24, and CD47.

105. The method of claim 102, wherein the immune cell is autologous or allogeneic and is selected from the group consisting of a T cell, a Natural Killer (NK) cell, a NKT cell, a B cell, a macrophage cell, an eosinophil or a neutrophil.


SEQUENCE LISTING (60 sequences)

SEQ ID NO: 1 - protein, 44 aa, Artificial Sequence
  Synthetic
RWPPSAACSGKESVVAIRTNSQSDFHLQTYGDEDLNELDPHYEM

SEQ ID NO: 2 - protein, 42 aa, Artificial
  Synthetic
RLKIQVRKAAITSYEKSDGVYTGLSTRNQETYETLKHEKPPQ

SEQ ID NO: 3 - protein, 86 aa, Artificial
  Synthetic
RWPPSAACSGKESVVAIRTNSQSDFHLQTYGDEDLNELDPHYEMRLKIQVRKAAITSYEKSDGVYTGLSTRNQETYETLKHEKPPQ

SEQ ID NO: 4 - DNA, 258 bp, Artificial
  Synthetic
cgctggcctccttctgcagcttgttcgggaaaagagtcagttgttgctataaggaccaatagccaatctgacttccacttacaaacttatggagatgaagatttgaatgaattagatcctcattatgaaatgcgactgaagatccaagtgcgaaaggcagctataaccagctatgagaaatcagatggtgtttacacgggcctgagcaccaggaaccaggagacttacgagactctgaagcatgagaaaccaccacag

SEQ ID NO: 5 - protein, 21 aa, Artificial
  Synthetic
MALPVTALLLPLALLLHAARP

SEQ ID NO: 6 - protein, 24 aa, Artificial
  Synthetic
IYIWAPLAGTCGVLLLSLVITLYC

SEQ ID NO: 7 - protein, 45 aa, Artificial
  Synthetic
TTTPAPRPPTPAPTIASQPLSLRPEACRPAAGGAVHTRGLDFACD

SEQ ID NO: 8 - protein, 383 aa, Artificial
  Synthetic
MGVLLTQRTLLSLVLALLFPSMASMAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 290 aa, Artificial
  Synthetic
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 10 - protein, 10 aa, Artificial
  Synthetic
GFTFSSYTMS

SEQ ID NO: 11 - protein, 17 aa, Artificial
  Synthetic
TISSRGTYTYYPDSVKG

SEQ ID NO: 12 - protein, 6 aa, Artificial
  Synthetic
EAIFTH

SEQ ID NO: 13 - protein, 11 aa, Artificial
  Synthetic
KASQDINNYHS

SEQ ID NO: 14 - protein, 7 aa, Artificial
  Synthetic
RANRLVD

SEQ ID NO: 15 - protein, 9 aa, Artificial
  Synthetic
LKYNVFPYT

SEQ ID NO: 16 - protein, 115 aa, Artificial
  Synthetic
QVQLLESGGGLVQPGGSLRLSCAASGFTFSSYTMSWVRQAPGQALEWMGTISSRGTYTYYPDSVKGRFTISRDNAKNSLYLQMNSLRAEDTAVYYCAREAIFTHWGRGTLVTVSS

SEQ ID NO: 17 - protein, 107 aa, Artificial
  Synthetic
DIQLTQSPSSLSASVGDRVTITCKASQDINNYHSWYQQKPGQAPRLLIYRANRLVDGVPDRFSGSGYGTDFTLTINNIESEDAAYYFCLKYNVFPYTFGQGTKVEIK

SEQ ID NO: 18 - protein, 237 aa, Artificial
  Synthetic
QVQLLESGGGLVQPGGSLRLSCAASGFTFSSYTMSWVRQAPGQALEWMGTISSRGTYTYYPDSVKGRFTISRDNAKNSLYLQMNSLRAEDTAVYYCAREAIFTHWGRGTLVTVSSGGGGSGGGGSGGGGSDIQLTQSPSSLSASVGDRVTITCKASQDINNYHSWYQQKPGQAPRLLIYRANRLVDGVPDRFSGSGYGTDFTLTINNIESEDAAYYFCLKYNVFPYTFGQGTKVEIK

SEQ ID NO: 19 - DNA, 22 bp, Artificial
  Synthetic
cagagcctcgcctttgccgatc

SEQ ID NO: 20 - DNA, 22 bp, Artificial
  Synthetic
catccatggtgagctggcggcg

SEQ ID NO: 21 - DNA, 20 bp, Artificial
  Synthetic
ggggcaagatggtaatgaag

SEQ ID NO: 22 - DNA, 20 bp, Artificial
  Synthetic
ccaggatactgagggcatgt

SEQ ID NO: 23 - DNA, 20 bp, Artificial
  Synthetic
gcacttcctccagaggtttg

SEQ ID NO: 24 - DNA, 20 bp, Artificial
  Synthetic
tcaccaggatgctcacattt

SEQ ID NO: 25 - DNA, 20 bp, Artificial
  Synthetic
gctgcactttggagtgatcg

SEQ ID NO: 26 - DNA, 20 bp, Artificial
  Synthetic
tcactcggggttcgagaaga

SEQ ID NO: 27 - DNA, 19 bp, Artificial
  Synthetic
cccagcatctgcaaagctc

SEQ ID NO: 28 - DNA, 20 bp, Artificial
  Synthetic
gtcaatgtacagctgccgca

SEQ ID NO: 29 - DNA, 20 bp, Artificial
  Synthetic
ggcttttcagctctgcatcg

SEQ ID NO: 30 - DNA, 21 bp, Artificial
  Synthetic
cgctacatctgaatgacctgc

SEQ ID NO: 31 - DNA, 20 bp, Artificial
  Synthetic
tctgagcagaccctggtaca

SEQ ID NO: 32 - DNA, 20 bp, Artificial
  Synthetic
gcaaggtaatgggggtcaca

SEQ ID NO: 33 - DNA, 21 bp, Artificial
  Synthetic
ccaaccacagcttcatgtgtc

SEQ ID NO: 34 - DNA, 20 bp, Artificial
  Synthetic
aaagcagtaggtcaggcagc

SEQ ID NO: 35 - DNA, 21 bp, Artificial
  Synthetic
acgacgtttccatcagcttgt

SEQ ID NO: 36 - DNA, 20 bp, Artificial
  Synthetic
tccaaggaatgtggtctggg

SEQ ID NO: 37 - DNA, 20 bp, Artificial
  Synthetic
tacccaccgccatactacct

SEQ ID NO: 38 - DNA, 20 bp, Artificial
  Synthetic
ctcagggtcttcgtggctca

SEQ ID NO: 39 - DNA, 21 bp, Artificial
  Synthetic
ccattccgctaggaaagacaa

SEQ ID NO: 40 - DNA, 22 bp, Artificial
  Synthetic
cctgtgttctctgtggactatg

SEQ ID NO: 41 - DNA, 22 bp, Artificial
  Synthetic
ctctagcagacagtgggatcta

SEQ ID NO: 42 - DNA, 20 bp, Artificial
  Synthetic
gaccttggctggtttgatga

SEQ ID NO: 43 - DNA, 20 bp, Artificial
  Synthetic
tgacgaattgtggatcggct

SEQ ID NO: 44 - DNA, 20 bp, Artificial
  Synthetic
ctggaccttggcttcgtgat

SEQ ID NO: 45 - DNA, 23 bp, Artificial
  Synthetic
gtagtctgctcaagatacacaga

SEQ ID NO: 46 - DNA, 20 bp, Artificial
  Synthetic
acaatctcccatgtgctgct

SEQ ID NO: 47 - DNA, 20 bp, Artificial
  Synthetic
tgccgttcacaagctcaagt

SEQ ID NO: 48 - DNA, 20 bp, Artificial
  Synthetic
actccaggtgtcagggtact

SEQ ID NO: 49 - DNA, 20 bp, Artificial
  Synthetic
ttgctccagctcctctatct

SEQ ID NO: 50 - DNA, 20 bp, Artificial
  Synthetic
gcctttggctttcacctttg

SEQ ID NO: 51 - DNA, 20 bp, Artificial
  Synthetic
gtggtgccgactacaagcga

SEQ ID NO: 52 - DNA, 21 bp, Artificial
  Synthetic
tttggaggatgtgccagaggt

SEQ ID NO: 53 - DNA, 23 bp, Artificial
  Synthetic
actcacctcttcagaacgaattg

SEQ ID NO: 54 - DNA, 23 bp, Artificial
  Synthetic
ccatctttggaaggttcaggttg

SEQ ID NO: 55 - DNA, 19 bp, Artificial
  Synthetic
ccaactgcttccccctctg

SEQ ID NO: 56 - DNA, 21 bp, Artificial
  Synthetic
tctgttacggtcaactcggtg

SEQ ID NO: 57 - protein, 15 aa, Artificial
  Synthetic
GGGGSGGGGSGGGGS

SEQ ID NO: 58 - protein, 6 aa, Artificial
  Synthetic
YGDEDL

SEQ ID NO: 59 - protein, 4 aa, Artificial
  Synthetic
YTGL

SEQ ID NO: 60 - protein, 4 aa, Artificial
  Synthetic
YETL